(19) United States
(12) Patent Application Publication
(10) Pub. No.: US 2017/0197996 A1
(43) Pub. Date: Jul. 13, 2017

(54) Cholinesterase Inhibitors

(71) Applicants: Charles M. Thompson, Missoula, MT (US); John M. Gerdes, Coos Bay, OR (US); Syed K. Ahmed, Hyderabad (IN); Yamina Belabassi, Missoula, MT (US)

(72) Inventors: Charles M. Thompson, Missoula, MT (US); John M. Gerdes, Coos Bay, OR (US); Syed K. Ahmed, Hyderabad (IN); Yamina Belabassi, Missoula, MT (US)

(73) Assignee: The University of Montana, Missoula, MT (US)

(21) Appl. No.: 15/258,356

(22) Filed: Sep. 7, 2016

Related U.S. Application Data
(63) Continuation of application No. 13/926,661, filed on Jun. 25, 2013, now abandoned.
(60) Provisional application No. 61/663,931, filed on Jun. 25, 2012.

(51) Int. Cl. C07F 9/40; C07F 9/38; A61K 51/04

(52) U.S. Cl. CPC C07F 9/4071; A61K 51/0489; C07F 9/38

(57) ABSTRACT

The invention provides compounds that inhibit cholinesterases, such as acetylcholinesterase and butyrylcholinesterase. Such compounds are useful to prevent or treat exposure of a patient (e.g., a human) to an organophosphoric nerve agent (e.g., sarin and VX) or to treat a patient suffering from a neurodegenerative disorder such as Alzheimer's Disease or Lewy Body Dementia. The compounds are further useful as diagnostic tools for use in medical or research radiography (e.g., positron emission tomography) when synthesized with a radionuclide (e.g., [18F]. Synthetic schemes to produce such compounds are also provided.

### CROSS-REFERENCE TO RELATED APPLICATIONS

This application is a continuation of U.S. Non-Provisional patent application Ser. No. 13/926,661, filed Jun. 25, 2013, which claims the benefit of the filing date of U.S. Provisional patent application Ser. No. 61/663,931, filed Jun. 25, 2012, the disclosures of which are incorporated by reference in their entirety.

### STATEMENT REGARDING FEDERALLY SPONSORED RESEARCH OR DEVELOPMENT

This invention was made with government support under Grant No. 1R21 NS072079 awarded by the National Institutes of Health. The government has certain rights in the invention.

### BACKGROUND OF THE INVENTION

The primary molecular target of organophosphorus nerve agents, whether weaponized or used as insecticides, is acetylcholinesterase (AChE). As nerve agents may inhibit other enzymes and because the receptors for acetylcholine (muscarinic and nicotinic acetylcholine receptors) are widely expressed, these agents can impact a number of biological processes. In addition to well-known CNS effects, vascular and inflammatory effects of nerve agents include vasoconstriction, an increase in blood brain barrier permeability, as well as modulation of lymphocyte proliferation and cytokine production. Exposure to organophosphates used in weapons or insecticides can result in significant morbidity or death. Present treatment of organophosphate poisoning consists of post-exposure intravenous or intramuscular administration of various combinations of drugs, including carbamates (e.g., pyridostigmine), anti-muscarinics (e.g., atropine), and ChE-reactivators such pralidoxime chloride (2-PAM). Novel inhibitors of acetylcholinesterase that block the binding of organophosphates can be used as prophylactic treatments for patients (e.g., a human, such as soldier or farmer) at risk for exposure to an organophosphate insecticide or chemical agent. It is further appreciated that organophosphate agents disrupt a number of biochemical and physiologic pathways, however these pathways remain unidentified or poorly understood. Consequently, there remains a significant need for novel and efficacious organophosphate compounds that can serve as pharmacological ligands and tissue biomarker agents.

### SUMMARY OF THE INVENTION

In general, the present invention is based on the discovery of compounds that exhibit cholinesterase (e.g., acetylcholinesterase) inhibitory activity. These compounds can be used to treat, prevent or simulate a disease, condition, or symptom in a patient (e.g., a human) resulting from cholinesterase dysregulation, such as certain neurodegenerative disorders from exposure to organophosphorus nerve agents. Accordingly, in a first aspect, the invention provides compounds presented and defined by the structural Formula I

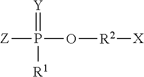

or a salt, ester or prodrug thereof, wherein
R¹ is selected from the group consisting of alkyl or aryl, wherein said alkyl or aryl may be branched or straight chain, substituted or unsubstituted;
R² is a branched, straight chain or cyclic alkyl;
X is selected from the group consisting of halogen, sulfonate ester, alkoxy, nitrile, azide, thiolether, sulfur ylide, amine, quaternary amine, and ester;
Y is selected from the group consisting of a lone pair of electrons, oxygen, and sulfur; and
Z is a leaving group selected from the group consisting of fluorine, nitrile, thiol, thiocholine, substituted phenols and heterols, alcohols, cholines, and substituted amines and amides.
   In one embodiment, substituent Z is any molecule that can be readily released from the phosphorus atom upon reaction with a nucleophile. In another embodiment, the compound is ethyl 4-nitrophenyl methylphosphonate; 2-fluoroethyl 4-nitrophenyl methylphosphonate; 2-chloroethyl 4-nitrophenyl methylphosphonate; 2-bromoethyl 4-nitrophenyl methylphosphonate; 2-iodoethyl 4-nitrophenyl methylphosphonate; 2-[(4-methylbenzenesulfonyl)oxy]ethyl 4-nitrophenyl methanephosphonate; ethyl 4-nitrophenyl ethylphosphonate; 2-fluoroethyl 4-nitrophenyl ethylphosphonate; 2-chloroethyl 4-nitrophenyl ethylphosphonate; 2-bromoethyl 4-nitrophenyl ethylphosphonate; 2-iodoethyl 4-nitrophenyl ethylphosphonate; 2-[(4-methylbenzenesulfonyl)oxy]ethyl 4-nitrophenyl ethane-1-phosphonate; 4-nitrophenyl propan-2-yl methylphosphonate; 1-fluoropropan-2-yl 4-nitrophenyl methylphosphonate; 1-chloropropan-2-yl 4-nitrophenyl methylphosphonate; 1-bromopropan-2-yl 4-nitrophenyl methylphosphonate; 1-iodopropan-2-yl 4-nitrophenyl methylphosphonate; 1-[(4-methylbenzenesulfonyl)oxy]propan-2-yl 4-nitrophenyl methanephosphonate; 4-nitrophenyl propan-2-yl ethylphosphonate; 1-fluoropropan-2-yl 4-nitrophenyl ethylphosphonate; 1-chloropropan-2-yl 4-nitrophenyl ethylphosphonate; 1-bromopropan-2-yl 4-nitrophenyl ethylphosphonate; 1-iodopropan-2-yl 4-nitrophenyl ethylphosphonate; 1-[(4-methylbenzenesulfonyl)oxy]propan-2-yl 4-nitrophenyl ethanephosphonate; 4-methylcyclohexyl 4-nitrophenyl methylphosphonate; 4-(fluoromethyl)cyclohexyl 4-nitrophenyl methylphosphonate; 4-(chloromethyl)cyclohexyl 4-nitrophenyl methylphosphonate; 4-(bromomethyl)cyclohexyl 4-nitrophenyl methylphosphonate; 4-(iodomethyl)cyclohexyl 4-nitrophenyl methylphosphonate; (4-{[methyl(4-nitrophenoxy)phosphoryl]oxy}cyclohexyl)methyl 4-methylbenzene-1-sulfonate; 4-methylcyclohexyl 4-nitrophenyl ethylphosphonate; 4-(fluoromethyl)cyclohexyl 4-nitrophenyl ethylphosphonate; 4-(chloromethyl)cyclohexyl 4-nitrophenyl ethylphosphonate; 4-(bromomethyl)cyclohexyl 4-nitrophenyl ethylphosphonate; 4-(iodomethyl)cyclohexyl 4-nitrophenyl ethylphosphonate; (4-{[ethyl(4-nitrophenoxy)phosphoryl]oxy}cyclohexyl)methyl 4-methylbenzene-1-sulfonate; ethyl 4-nitrophenyl methyl(sulfanylidene)phosphonite; 2-fluoroethyl 4-nitrophenyl methyl(sulfanylidene)phosphonite; 2-chloroethyl 4-nitrophenyl methyl(sulfanylidene)phosphonite; 2-bromoethyl 4-nitrophenyl methyl(sulfanylidene)phosphonite; 2-iodoethyl 4-nitrophenyl methyl(sulfanylidene)phosphonite; or 2-[(4-methylbenzenesulfonyl)oxy]ethyl 4-nitrophenyl P-methylsulfanephosphonite. In a further embodiment, substituent R¹ contains a radionuclide, such as fluorine-18, carbon-11, nitrogen-13, oxygen-15, bromine-76, or iodine-124. In another embodiment, the compound is combined with a pharmaceutically acceptable excipient.

In a second aspect, the invention provides a method of synthesizing a compound defined and presented by structural Formula I, or a salt, ester or prodrug thereof, by reacting an alkylphosphonic dichloride with two equivalents of p-nitrophenol in the presence of a base to form a bis[p-nitrophenoxy] alkylphosphonate, performing monohydrolysis to yield a p-nitrophenoxy alkylphosphonic acid, and reacting the p-nitrophenoxy alkylphosphonic acid with a carbodiimide or a coupling reagent and a substituted alcohol to yield a compound of the invention.

In a third aspect, the invention provides a method of synthesizing a tracer compound defined and presented by structural Formula I, or a salt, ester or prodrug thereof, wherein R¹ further includes a radionuclide, by reacting an alkylphosphonic dichloride with two equivalents of p-nitrophenol in the presence of a base to form a bis[p-nitrophenoxy] alkylphosphonate, performing monohydrolysis to yield a p-nitrophenoxy alkylphosphonic acid, and reacting the p-nitrophenoxy alkylphosphonic acid with cesium carbonate and radiolabeled [¹⁸F] beta-fluoroethyltosylate to yield a tracer compound of the invention. In one embodiment, the radionuclide is fluorine-18. In another embodiment, the radionuclide is carbon-11, nitrogen-13, oxygen-15, bromine-76, or iodine-124.

In a fourth aspect, the invention includes a method of treating a patient by administering to the patient a compound defined and presented by structural Formula I, or a salt, ester or prodrug thereof. In one embodiment, the administered compound further includes a radionuclide, such as fluorine-18. In another embodiment, the radionuclide is carbon-11, nitrogen-13, oxygen-15, bromine-76, or iodine-124.

In a fifth aspect, the invention includes a method of detecting a cholinesterase by contacting a tracer compound defined and presented by structural Formula I, or a salt, ester or prodrug thereof, wherein R1 wherein R¹ further includes a radionuclide, with a cholinesterase and detecting the tracer compound with a radiographic scanner. In one embodiment, the radiographic scanner is a positron emission tomography (PET) scanner.

Further embodiments, features, and advantages of the present invention, as well as the structure and operation of the various embodiments of the present invention, are described in detail below with a reference to the accompanying drawings.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, the singular form “a,” “an,” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the terms below have the meanings indicated.

The term “acyl” as used herein, alone or in combination, refers to a carbonyl attached to an alkenyl, alkyl, aryl, cycloalkyl, heteroaryl, heterocyclyl, or any other moiety where the atom attached to the carbonyl is carbon. An “acetyl” group refers to a —C(O)CH₃ group.

An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl” as used herein, alone or in combination, refers to a straight-chain or branched-chain hydrocarbon radical having one or more double bonds optionally substituted and containing from 2 to 20, preferably 2 to 6, carbon atoms. Alkenyl refers to a carbon-carbon double bond system attached at two or more positions such as ethenylene [(—CH═CH—), (—C::C—)]. Examples of alkenyl radicals include ethenyl, propenyl, 2-methylpropenyl, 1,4-butadienyl and the like.

The term “alkoxy” as used herein, alone or in combination, refers to an alkyl ether radical, optionally substituted wherein the term alkyl is as defined below. Examples of alkyl ether radicals include methoxy, ethoxy, n-propoxy, isopropoxy, n-butoxy, iso-butoxy, sec-butoxy, tert-butoxy, and the like.

The term “alkyl” as used herein, alone or in combination, refers to a straight-chain or branched-chain alkyl radical optionally substituted containing from 1 to 20 and including 20, preferably 1 to 10, and more preferably I to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl and the like.

The term “alkylamino” as used herein, alone or in combination, refers to an alkyl group optionally substituted attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The term “alkylthio” as used herein, alone or in combination, refers to an alkyl thioether (R—S—) radical wherein the term alkyl is as defined above and wherein the sulfur may be singly or doubly oxidized. Examples of alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynyl” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The term “amido” as used herein, alone or in combination, refer to an amino group as described below attached to the parent molecular moiety through a carbonyl group, or vice versa.

The term “amino” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl” as used herein, alone or in combination, means a carbocyclic aromatic system containing one, two or three rings wherein such rings may be attached together in a pendent manner or may be fused optionally substituted with at least one halogen, an alkyl containing from 1 to 3 carbon atoms, an alkoxyl, an aryl radical, a nitro function, a polyether radical, a heteroaryl radical, a benzoyl radical, an alkyl ester group, a carboxylic acid, a hydroxyl optionally protected with an acetyl or benzoyl group, or an amino function optionally protected with an acetyl or benzoyl group or optionally substituted with at least one alkyl containing from 1 to 12 carbon atoms.

The terms “arylalkyl” or “aralkyl” as used herein, alone or in combination, refers to an aryl group attached to the parent molecular moiety through an alkyl group.

The term “aryloxy” as used herein, alone or in combination, refers to an aryl group attached to the parent molecular moiety through an oxygen atom.

The term “polyether radical” means a polyether radical containing from 2 to 6 carbon atoms interrupted with at least one oxygen atom, such as methoxymethyl, ethoxymethyl or methoxyethoxymethyl radicals or methoxyethyl.

The terms “benzo” and “benz” as used herein, alone or in combination, refer to the divalent radical C₆H₄═ derived from benzene. Examples include benzothiophene and benzimidazole.

The terms “carbamate” and “carbamoyl” as used herein, alone or in combination, refers to an ester of carbamic acid (—NHCOO—) which may be attached to the parent molecular moiety from either the nitrogen or acid end, and which may be optionally substituted as defined herein.

The term “carbonyl” as used herein, when alone includes formyl [—C(O)H] and in combination is a —C(O)— group.

The term “carboxy” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano” as used herein, alone or in combination, refers to —CN.

The term “cycloalkyl” or, alternatively, “carbocycle”, as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo-fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester” as used herein, alone or in combination, refers to a carboxy group bridging two moieties linked at carbon atoms.

The term “ether” as used herein, alone or in combination, refers to an oxygen atom bridging two moieties linked at carbon atoms.

The terms “halo” or “halogen” as used herein, alone or in combination, refers to fluorine, chlorine, bromine, or iodine.

The term “haloalkyl” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difiuoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CHF—), difluoromethylene (—CF₂—), chloromethylene (—CHCl—) and the like.

The term “heteroalkyl” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocyclyl”, as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocyclyl” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocyclyl groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocyclyl groups may be optionally substituted unless specifically prohibited.

The term “hydroxyl” as used herein, alone or in combination, refers to —OH.

The phrase “in the main chain” refers to the longest contiguous or adjacent chain of carbon atoms starting at the point of attachment of a group to the compounds of this invention.

The phrase “linear chain of atoms” refers to the longest straight chain of atoms independently selected from carbon, nitrogen, oxygen and sulfur.

The term “lower” as used herein, alone or in combination, means containing from 1 to and including 6 carbon atoms.

The term “negatively-charged ion” as used herein, refers to any negatively-charged ion or molecule, either inorganic (e.g., Cl⁻, Br⁻, I⁻) or organic (e.g., TsO— (i.e., tosylate)).

The term “nitro” as used herein, alone or in combination, refers to —NO₂.

The term “perhaloalkyl” as used herein, alone or in combination, refers to an alkyl group where all of the hydrogen atoms are replaced by halogen atoms.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstitutedsilyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

Asymmetric centers exist in the compounds of the present invention. These centers are designated by the symbols “R” or “S,” depending on the configuration of substituents around the chiral carbon or phosphorus atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Optical isomers are compounds with the same molecular formula but differ in the direction they rotate plane polarized light. There are two types of optical isomers. The first type of optical isomers are compounds that are mirror images of one another but cannot be superimposed on each other. These isomers are called “enantiomers.” The second type of optical isomers are molecules that are not mirror images but each molecule rotates plane polarized light and are considered optically-active. Such molecules are called “diastereoisomers.” Diasteroisomers differ not only in the way they rotate plane polarized light, but also their physical properties. The term “optical isomer” comprises more particularly the enantiomers and the diastereoisomers, in pure form or in the form of a mixture.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “imaging agent” as used herein refers to any moiety useful for the detection, tracing, or visualization of a compound of the invention when coupled thereto. Imaging agents include, e.g., an enzyme, a fluorescent label (e.g., fluorescein), a luminescent label, a bioluminescent label, a magnetic label, a metallic particle (e.g., a gold particle), a nanoparticle, an antibody or fragment thereof (e.g., a Fab, Fab′, or F(ab′)₂ molecule), and biotin. An imaging agent can be coupled to a compound of the invention by, for example, a covalent bond, ionic bond, van der Waals interaction or a hydrophobic bond. An imaging agent of the invention can be a radiolabel coupled to a compound of the invention, or a radioisotope incorporated into the chemical structure of a compound of the invention. Methods of detecting such imaging agents include, but are not limited to, positron emission tomography (PET), X-ray computed tomography (CT) and magnetic resonance imaging (MRI).

The term “nerve agent” as used herein, refers to any toxic chemical that disrupts the function of neurons, specifically the transduction of action potentials. Nerve agents have historically been weaponized or used as insecticides. Common nerve agents are organophosphates, including but not limited to, diisopropylfluorophosphate (DFP), GA (tabun), GB (sarin), GD (soman), CF (cyclosarin), GE, CV, YE, VG (amiton), VM, VR (RVX or Russian VX), VS, and VX. Other chemical warfare agents of interest are phosphonothioic acid, methyl-, S-(2-bis(1-methylethylamino)-ethyl) O-ethyl ester O-ethyl; S-(2-diisopropylaminoethyl) methylphosphonothiolate; S-2-diisopropylaminoethyl O-ethyl methylphosphonothioate; S-2((2-diisopropylamino)ethyl) O-ethyl methylphosphonothiolate; O-ethyl S-(2-diisopropylaminoethyl) methylphosphonothioate; O-ethyl S-(2-diisopropylaminoethyl) methylthiolphosphonoate; S-(2-diisopropylaminoethyl) O-ethyl methyl phosphonothiolate; ethyl-5-dimethylaminoethyl methylphosphonothiolate VX EA 1701; and TX60.

The term “neurodegenerative disorder” as used herein, refers to any disease, disorder, condition, or symptom characterized by the structural or functional loss of neurons. Neurodegenerative disorders include, e.g., Alzheimer's disease, Parkinson's disease, Huntington's Disease, Lewy Body dementia, and amyotrophic lateral sclerosis (ALS).

The phrase “therapeutically effective” is intended to qualify the amount of active ingredients used in the treatment of a disease or disorder. This amount will achieve the goal of reducing or eliminating the disease or disorder.

The term “therapeutically acceptable” refers to those compounds (or salts, esters, prodrugs, tautomers, zwitterionic forms, etc. thereof) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “patient” does not denote a particular age or sex.

The term “prodrug” refers to a compound that is made more active in vivo. Certain compounds of the present invention may also exist as prodrugs, as described in Hydrolysis in Drug and Prodrug Metabolism: Chemistry, Biochemistry, and Enzymology, Testa, Bernard and Wiley-VHCA, Zurich, Switzerland 2003. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bio-available by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug is a compound that is administered as an ester (the “prodrug”), but then is metabolically hydrolyzed to the carboxylic acid, the active entity. Additional examples include peptidyl derivatives of a compound.

The compounds of the invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Stahl, P. Heinrich, Pharmaceutical Salts: Properties, Selection, and Use, Wiley-VCHA, Zurich, Switzerland (2002).

The term “therapeutically acceptable salt” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

### BRIEF DESCRIPTION OF THE DRAWINGS

FIG. 1 is a schematic showing the chemical synthesis of several analogs of the invention, including compounds 6, [¹⁸F]-6, and compounds 9.

FIG. 2 is a graph showing that compound 6 (R═Me, n=1, X═F) binds to and inhibits acetylcholinesterase. A time-dependent block of human recombinant acetylcholinesterase enzyme activity occurs over a period of 6 minutes.

FIG. 3 is a graph showing the acetylcholinesterase inhibition kinetics of compound 6 (R═Me, n=1, X═F). Acetylcholinesterase enzymatic activity (control corrected) is plotted versus the amount of time the inhibitor was incubated with enzyme to yield a slope that correlates with bimolecular inhibitor constant (kᵢ) when corrected for the inhibitor concentration. In this instance, the kᵢ for compound 6 is 3.45×105M⁻¹min⁻¹ representing a strong inhibitor. This value is comparable to a number of well known organophosphorus inhibitors acting on this enzyme type.

FIG. 4 is a schematic showing the chemical reactions leading to ¹⁸F positron-labeled acetylcholinesterase.

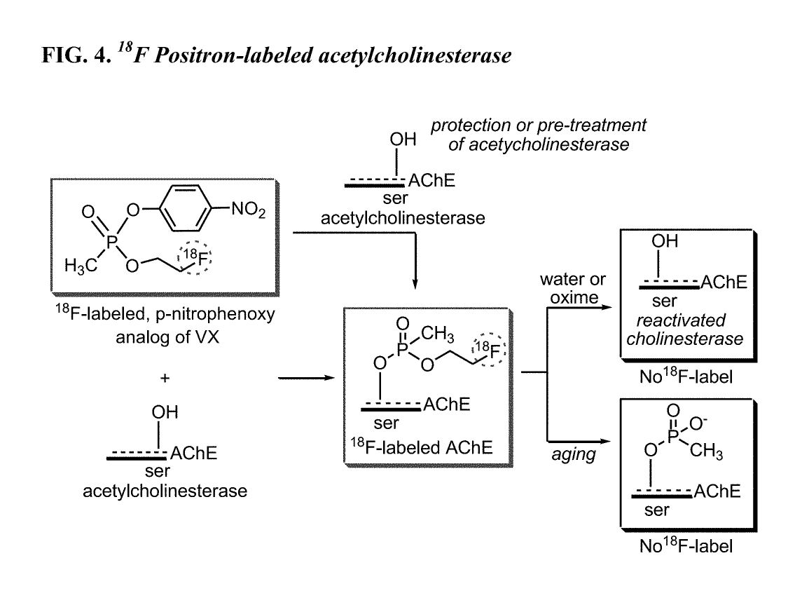

FIG. 5 is a schematic showing mechanisms of AChE inhibition of a ¹⁸F positron-labeled analog of VX (red) to afford a similar adduct as VX.

FIG. 6 is a schematic showing the synthetic scheme to produce radiolabeled ¹⁸F-phosphonates.

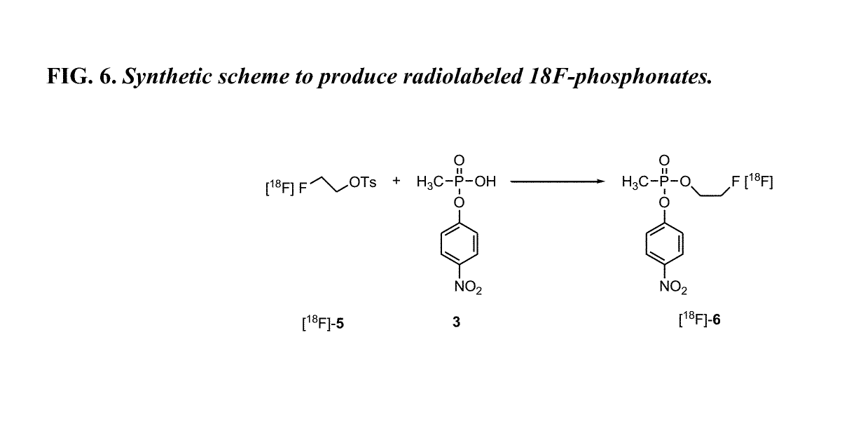

FIG. 7 is a table showing the inhibition of acetylcholinesterases by exemplary compounds of the invention.

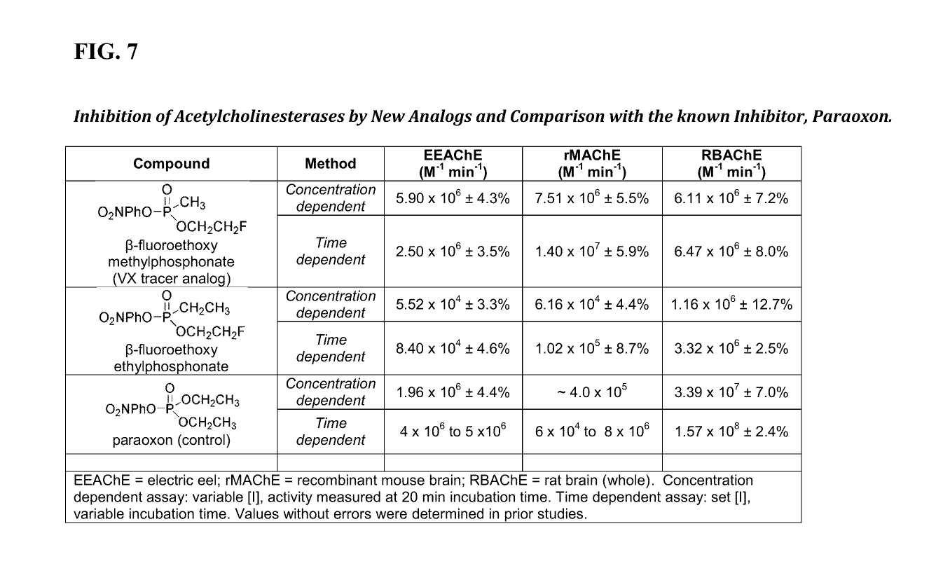

FIG. 8 is a graph showing the baseline PET tracer-activity of compound [¹⁸F]-6 in rat brain (Example IX, Trial A), in which the central nervous tissue regions of interest are defined as: FrCtx as frontal cortex, MotCtx as motor cortex, CgCtx as cingulate cortex, CP as caudate-putamen, TH as thalamus, ME as mescencephalon referred to as the mid-brain, RE as the rhombencephalon otherwise referred to as the brain stem, and CE as cerebellum.).

FIG. 9 is a graph showing the PET tracer-activity of compound [¹⁸F]-6 following pre-challenge with compound 6 (unlabeled) in rat brain (Example IX, Trial B), in which the central nervous tissue regions of interset are defined as: FrCtx as frontal cortex, MotCtx as motor cortex, CgCtx as cingulate cortex, CP as caudate-putamen, TH as thalamus, ME as mescencephalon referred to as the mid-brain, RE as the rhombencephalon otherwise referred to as the brain stem, and CE as cerebellum.).

FIG. 10 is a graph showing a comparison of the PET tracer-activity of compound [¹⁸F]-6 in select rat peripheral tissues, including lung and liver tissue.

FIG. 11 is a graph showing the PET tracer-activity of compound [¹⁸F]-6 following pre-challenge with the known organophosphorus compound paraoxon in rat brain (Example IX, Trial C), in which the central nervous tissue regions of interest are defined as: FrCtx as frontal cortex, MotCtx as motor cortex, CgCtx as cingulate cortex, CP as caudate-putamen, TH as thalamus, ME as mescencephalon referred to as the mid-brain, RE as the rhombencephalon otherwise referred to as the brain stem, and CE as cerebellum.).

FIG. 12 is a graph showing the PET tracer-activity of compound [¹⁸F]-6 following pre-challenge with the known organophosphorus compound echothiophate in rat brain (Example IX, Trial D), in which the central nervous tissue regions of interest are defined as: FrCtx as frontal cortex, MotCtx as motor cortex, CgCtx as cingulate cortex, CP as caudate-putamen, TH as thalamus, ME as mescencephalon referred to as the mid-brain, RE as the rhombencephalon otherwise referred to as the brain stem, and CE as cerebellum.).

### DETAILED DESCRIPTION OF THE INVENTION

The potential deployment of organophosphate nerve agents in terrorist or military actions is of immediate concern and has prompted new investigations to develop therapeutics to alleviate or cure human exposures. The present invention features novel organophosphate compounds that are effective inhibitors of cholinesterase enzymes, such as acetylcholinesterase (AChE) and pseudocholinesterase (BChe). The inhibitory properties of the compounds of the invention can therefore be used to treat, prevent, simulate or visualize diseases, disorders, conditions, or symptoms in a patient (e.g., a human) that involve, directly or indirectly, acetylcholine or butyrylcholine metabolism, either caused naturally (e.g., neurodegenerative disorder) or by exposure to an artificial agent (e.g., a nerve agent).

### Compounds of the Invention

The compounds of the invention are represented by the following Formula I:

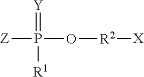

or a salt, ester or prodrug thereof, wherein
R¹ is selected from the group consisting of alkyl or aryl, wherein said alkyl or aryl may be branched or straight chain, substituted or unsubstituted;
R² is a branched, straight chain or cyclic alkyl;
X is selected from the group consisting of halogen, sulfonate ester, alkoxy, nitrile (CN), azide (N₃), thiolether (e.g., SR), sulfur ylide (e.g., SR′R″, wherein R′ and R″ are individually an alkyl or aryl group, branched or straight chain, substituted or unsubstituted, and wherein S has a positive charge), amine (e.g., NR′R″, wherein R′ and R″ are an alkyl or aryl group, branched or straight chain, substituted or unsubstituted), quaternary amine (e.g., NR′R″R*, wherein R′, R″, and R* are individually an alkyl or aryl group, branched or straight chain, substituted or unsubstituted), and ester (e.g., OC(O)R′ or C(O)OR′, wherein R′ is an alkyl or aryl group, branched or straight chain, substituted or unsubstituted);
Y is selected from the group consisting of a lone pair of electrons, oxygen, and sulfur; and
Z is a leaving group selected from the group consisting of fluorine, CN, thiol, thiocholine, substituted phenols and heterols, alcohols, cholines, and substituted amines and amides.
   In one embodiment of the invention, substituent Z is any atom or molecule that can be readily released from the phosphorus atom upon reaction with a nucleophile.

The compounds of the invention are useful for treating, simulating, visualizing or diagnosing diseases, disorders, and symptoms characterized by or related to organophosphate poisoning caused by, for example, exposure to a nerve agent. The compounds of the invention are also useful for the analysis of neurodegenerative disorders such as, e.g., amyotrophic lateral sclerosis (ALS), Huntington's disease, and Parkinson's disease. Furthermore, the compounds of the invention are useful in diagnostic or research imaging applications (e.g., in vivo, in vitro, and ex vivo), such as the imaging of acetylcholinesterase by radiography (e.g., positron emission tomography (PET)), to evaluate the function and distribution of the enzyme in a patient (e.g., a human), or organ/tissue sample (e.g., a biopsy).

The compounds of the invention offer several advantages over other cholinesterase inhibitors known in the art. First, inhibition of acetylcholinesterase through inactivation by covalent bond formation by other compounds have demonstrated different acetylcholinesterase potency and kinetic profiles, relative to the compounds of the invention; where the latter are considered more useful for in vivo interactions with acetylcholinesterase either alone or in the presence of other organophosphorus agents (e.g., nerve agents). Second, compounds derived from Formula I (e.g., compounds 6) are novel and potent acetylcholinesterase inhibitors. Significantly, the O—R2-X side chain group of the compounds of the invention has not been previously described particularly for the purpose of isotope installation. Finally, radiolabeled tracer compounds derived Formula I inhibitor (e.g., compound [¹⁸F]-6) are able to inactivate acetylcholinesterase by covalent attachment and thus, are considered of greater value for detecting acetylcholinesterase in various tissues by way of dynamic imaging and/or through autoradiography.

### Nerve Agents

One or more compounds of the invention can also be used to prevent or treat exposure of a patient (e.g., a human) to a nerve agent, such as an organophosphate-based chemical weapon or insecticide. Such treatment or prophylaxis serves to competitively bind cholinesterase (e.g., AChE) molecules in a patient (e.g., a human) against organophosphate nerve agents. The preferential binding of cholinesterase molecules (e.g., AChE) by the compounds of the invention can prevent, reduce, or treat the symptoms or conditions typically suffered by a patient (e.g., a human) upon exposure to organophosphate nerve agents.

The compounds of the invention can be particularly useful for the prophylactic treatment of members of armed forces at risk of exposure to nerve agents, especially weaponized nerve agents, in the course of duty. Similarly, the compounds of the invention can be used to treat or prophylax a patient (e.g., a human) or animal that may be exposed to organophosphate nerve agents, such as insecticides, during the course of employment (e.g., agricultural workers or livestock).

### Neurodegenerative Disorders

One or more compounds of the invention can be used to treat a patient (e.g., a human) at risk of developing or already suffering from a neurodegenerative disorder, such as Alzheimer's Disease (“AD”) or Lewy Body Dementia, in which inhibition of a cholinesterase (e.g., AChE) delays, stops, or reverses disease progression or partially or completely alleviates the symptoms (e.g., memory loss) of the neurodegenerative disease.

### Methods of Prevention and Treatment

The compounds and methods of the invention can be used, alone or in combination with other agents and compounds, to treat a patient (e.g., a human) that suffers from or is at risk of suffering from a disease, disorder, condition or symptom described herein (e.g., nerve agent exposure or a neurodegenerative disorder). The compounds of the invention can further be used, alone or in combination with other agents and compounds, to simulate, visualize, diagnose or prevent the development of a disease, disorder, condition or symptom associated with cholinesterase metabolism. Each such treatment described above includes the step of administering to a patient in need thereof a therapeutically effective amount of the compound of the invention described herein to delay, reduce or prevent such disease, disorder, condition, or symptom.

Besides being useful for human treatment, the compounds and formulations of the present invention are also useful for the treatment of animals, e.g., the veterinary treatment of domesticated animal, companion animals (e.g., dogs and cats), exotic animals, farm animals (e.g., ungulates, including horses, cows, sheep, goats, and pigs), and animals used in scientific research (e.g., rodents and non-human primates).

### Methods of Research Use

The compounds of the invention can be used as research tools to assess the mechanism of biological action of organophosphate compounds including agents that block, interrupt or inhibit the activity of cholinesterases, related serine hydrolases, and other potential target proteins. For example, a compound derived from Formula I can be used to assess potential treatments, antidotes and ameliorating agents intended to reverse the effect of organophosphorus chemical threat agents, insecticides, or other organophosphates considered toxic to mammals.

### Methods of Diagnostic Imaging

The invention further features compounds and methods useful for in vivo or in vitro radiographic imaging studies of cholinesterase metabolism in a patient (e.g., a human). Compounds of the invention that contain a radionuclide, such as fluorine-18, can also be used, alone or in combination with other agents and compounds, in radiographic medical imaging applications to diagnose or follow the progression of diseases, disorders, conditions or symptoms related to cholinesterase metabolism in a patient (e.g., a human). Such imaging agents or “tracer compounds” can further be used, for example, in functional assays to assess the efficacy of new and existing countermeasures to organophosphate exposure.

The inventors have discovered novel processes of adding a radionuclide (e.g., fluorine-18) to any compound represented by Formula I to yield a radiographic tracer compound. The discovery of these compounds and related processes represent an unexpected advance in the art of radiotracer chemistry as fluorine-18 complexes are notoriously difficult to synthesize (see, e.g., Lee et al., A Fluoride-Derived Electrophilic Late-State Fluorination Reagent for PET Imaging, Science 334:639 (2011)). The addition of such a radionuclide tag (e.g., fluorine-18) to the genus of acetylcholinesterase inhibitors described herein yields a “tracer” compound that can be imaged using, e.g., a positron emission tomography (PET) scanner. In one embodiment of the invention, a tracer compound of the invention can be used to study acetylcholinesterase activity. Diagnostic imaging studies using these tracer compounds of the invention can be performed in vivo or in patient biopsies and tissue samples ex vivo. Similarly, the tracer compounds of the invention are also useful for the in vitro or ex vivo study of acetylcholinesterase activity for biomedical research purposes.

Radiologists and other medical clinicians are skilled in the use of radiographic imaging devices, such as positron emission tomography (PET) scanners, and methods of imaging tracer compounds, such as the radionuclide compounds of the invention, in a patient are widely known (e.g., Saha, Basics of PET Imaging: Physics, Chemistry, and Regulations, Springer (2010) ISBN 978-1-4419-0804-9, hereby incorporated by reference).

The radionuclide compounds and formulations of the present invention are also useful for the medical imaging of animals, e.g., the veterinary treatment of domesticated animal, companion animals (e.g., dogs and cats), exotic animals, farm animals (e.g., ungulates, including horses, cows, sheep, goats, and pigs), and animals used in scientific research (e.g., rodents and non-human primates).

### Methods of Radionuclide Compound Synthesis

The radionuclide tracer compounds of the invention can be synthesized by several techniques known to persons skilled in the art. For example, for the substitution of a carbon atom by a carbon-11, several derivatives such as [¹¹C]methyl iodide or [¹¹C]methyl triflate (Welch M. J. et al. (2003) In Handbook of Radiopharmaceuticals—Radiochemistry and Applications (Welch M J, Redvanly C S Eds.), New York-Chichester-Brisbane-Toronto, Wiley-Interscience Pub., 1-848).

In the case of a labeling with fluorine-18, the radioisotope may be directly attached to a core structure by nucleophilic aliphatic or aromatic (including heteroaromatic (Dolle F. et al. (2005) Curr. Pharm. Design 11: 3221-3235)) substitutions or electrophilic substitutions or linked through the addition of a spacer group, both techniques known to persons skilled in the art (Kilbourn M R. (1990) In fluorine-18 Labeling of Radiopharmaceuticals, Nuclear Science Series (Kilbourn M R Ed.), National Academy Press, Washington, D.C., 1-149; Lasne M.-C. et al. (2002) Topics in Current Chemistry 222: 201-258; Cai L. et al. (2008) Eur. J. Org. Chem. 17: 2853-2873; Dolle F. et al. (2008) In Fluorine and Health: Molecular Imaging, Biomedical Materials and Pharmaceuticals, Tressaud A, Haufe G (Eds). Elsevier: Amsterdam-Boston-Heidelberg-London-New York-Oxford-Paris-San Diego-San Francisco-Singapore-Sydney-Tokyo, 3-65). An alkyl, alkenyl or alkynyl linker may also be used for the addition of the fluorine-18 atom (Damont A. et al. (2008) J. Label. Compds Radiopharm. 51: 286-292; Dolle F. et al., (2006) Bioorg. Med. Chem. 14: 1115-1125; Dolle F. et al. (2007) J. Label. Compds Radiopharm. 50: 716-723). Additional methods of producing radionuclide (e.g., fluorine-18) labeled compounds are described in U.S. Patent Application Publications No. 2006/0100465, 2010/0292478, and 2011/0184159, each hereby incorporated by reference.

In the case of a labeling with other halogens (e.g., bromine-76, iodine-123 or iodine-124), the radioisotope may also be directly attached by nucleophilic or electrophilic substitutions to a core structure or linked through the addition of a spacer group, both techniques known to persons skilled in the art (Maziere et al. Curr. Pharm. Des. 7:1931-1943 (2001); Coenen et al., “In Radioiodination reactions for pharmaceuticals—Compendium for effective synthesis strategies,” Coenen H. H., Mertens J., Maziere B. (Eds), Springer Verlag, Berlin-Heidelberg, 1-101 (2006)).

In the case of the labeling with metal radioisotopes (e.g., gallium-68, zinc-62, copper-62, copper-64, gallium-68, germanium-68, strontium-82, rubidium-82, technicium-94m, or technetium-99m), the preferred approach used, which will be considered by a person skilled in the art, is the use of a bifunctional chelating agent based on, for example, the open-chain polyaminocarboxylates ethylenediamine tetraacetic acid (EDTA) and diethylenetriamine pentaacetic acid (DTPA), the polyaminocarboxylic macrocycle 1,4,7,10-tetraazacyclododecane-1,4,7,10-tetraacetic acid (DOTA), mercaptoacetyldi- and triglycine (MAG2, MAG3), bis-(S-benzoyl-thioglycoloyl)diaminopropanoate ((SBT)₂DAP) and hydrazinonicotinic acid (HYNIC), facilitating the complexation of the radiometal cation at one function and the covalent attachment to a core molecule at another (Brunner U. K. et al. (1995) Radiotracer production—Radiometals and their chelates In Principle of Nuclear Medicine, Wagner H. N. (Ed). Saunders: Philadelphia, 220-228; Weiner R. E. et al. (2003) Chemistry of gallium and indium radiopharmaceuticals In Handbook of Radiopharmaceuticals—Radiochemistry and Applications (Welch M J, Redvanly C S Eds.), New York-Chichester-Brisbane-Toronto, Wiley-Interscience Pub., 363-400; Anderson C. J. et al. (2003) Chemistry of copper radionucleides and radiopharmaceutical products In Handbook of Radiopharmaceuticals—Radiochemistry and Applications (Welch M J, Redvanly C S Eds.), New York-Chichester-Brisbane-Toronto, Wiley-Interscience Pub., 401-422; Mahmood A. et al. (2003) Technetium radiopharmaceuticals In Handbook of Radiopharmaceuticals—Radiochemistry and Applications (Welch M J, Redvanly C S Eds.), New York-Chichester-Brisbane-Toronto, Wiley-Interscience Pub., 323-362).

Further methods of synthesizing the compounds of the invention are described below in the Examples.

### Radionuclide Specific Activity

The tracer compounds of the invention described herein that include a radionuclide (e.g., fluorine-18) can be synthesized to adjust the specific activity of the compound. Specific activity is defined as the radioactivity per unit mass of a radionuclide or a labeled compound. For example, if a 50 mg sample contains 100 mCi (370 MBq), then the specific activity of the sample is given as 100/50=2 mCi/mg or 74 MBq/mg. Specific activity should not be confused with the concentration of a compound containing a radionuclide, which are generally expressed in mCi/mL or MBq/mL. The specific activity is an important parameter to consider in radiolabeling and in vivo biodistribution of tracers, such as the radionuclide compounds of the invention. Cold molecules in low specific activity radiopharmaceuticals compete with radioactive molecules and lower the uptake of the tracer in the target tissue(s). Similarly, low specific activity radionuclides yield poor radiolabeling, and hence, poor radiography (e.g., PET). For these reasons, the tracer compounds of the invention containing fluorine-18 are synthesized having a specific activity of at least 1.0, 1.2, 1.4, 1.8, 2.0, 2.2, 2.4, or 2.6 Ci/mmol. In one embodiment of the invention, the fluorine-18 tracer compound has a specific activity of at least 1.0 Ci/mmol.

Persons having skill in the art are aware of methods that can increase or decrease the specific activity of a desired radionuclide compound of the invention. For example, electrophilic fluorination of palladium aryl complexes can be used to yield tracer compounds of the invention containing fluorine-18 with high specific activity (Lee et al., “A Fluoride-Derived Electrophilic Late-State Fluorination Reagent for PET Imaging,” Science 334:639 (2011), hereby incorporated by reference).

### Compound Administration and Formulation

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid. The novel compounds described herein can be prepared in a form of pharmaceutically acceptable salts that will be prepared from nontoxic inorganic or organic bases including but not limited to aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally-occurring substituted amines, cyclic amines, and basic ion exchange resins, such as arginine, betaine, caffeine, choline, ethylamine, 2-diethylaminoethano, 1,2-dimethylaminoethanol, ethanolamine, ethylenediamine, N-ethyl-morpholine, N-ethylpiperidine, glucamine, glucosamine, histidine, hydroxylamine, isopropylamine, lysine, methylglucamine, morpholine, piperazine, piperidine, polyamine resins, procaine, purines, theobromine, triethylamine, trimethylamine, tri shydroxylmethyl amino methane, tripropyl amine, and tromethamine.

If the compounds of the invention are basic, salts could be prepared in a form of pharmaceutically acceptable salts that will be prepared from nontoxic inorganic or organic acids including but not limited to hydrochloric, hydrobromic, phosphoric, sulfuric, tartaric, citric, acetic, fumaric, alkylsulphonic, naphthalenesulphonic, para-toluenesulphonic, camphoric acids, benzenesulfonic, benzoic, camphorsulfonic, citric, ethanesulfonic, gluconic, glutamic, isethonic, lactic, maleic, malic, mandelic, methanesulfonic, mucic, nitric, pamoic, pantothenic, phosphoric, and succinic.

While it may be possible for the compounds of the invention to be administered as the raw chemical, it is also possible to present them as a pharmaceutical formulation. Accordingly, the present invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. When used in the diagnostic imaging methods of the invention, the compounds of the invention are preferably administered to the patient (e.g., a human) by intravenous injection. The formulations may conveniently be presented in unit dosage form and may be prepared by any of the methods well known in the art of pharmacy. All methods include the step of bringing into association a compound of the present invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of the invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compounds of the invention may take the form of tablets, lozenges, pastilles, or gels formulated in conventional manner. Such compositions may comprise the active ingredient in a flavored basis such as sucrose and acacia or tragacanth.

The compounds of the invention may also be formulated in rectal compositions such as suppositories or retention enemas, e.g., containing conventional suppository bases such as cocoa butter, polyethylene glycol, or other glycerides.

Compounds of the invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include solid, liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Via the topical route, the pharmaceutical composition according to the invention may be in the form of liquid or semi liquid such as ointments, or in the form of solid such as powders. It may also be in the form of suspensions such as polymeric microspheres, or polymer patches and hydrogels allowing a controlled release. This topical composition may be in anhydrous form, in aqueous form or in the form of an emulsion. The compounds are used topically at a concentration generally of between 0.001% and 10% by weight and preferably between 0.01% and 1% by weight, relative to the total weight of the composition.

For administration by inhalation, the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

Compounds according to the invention can be administered at a daily dose of about 0.001 mg/kg to 100 mg/kg of body weight, in 1 to 3 dosage intakes. Further, compounds can be used systemically, at a concentration generally of between 0.001% and 10% by weight and preferably between 0.01% and 1% by weight, relative to the weight of the composition.

The amount of active ingredient that may be combined with the carrier materials to produce a single dosage form will vary depending upon the host treated and the particular mode of administration.

The compounds of the invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds of the invention described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the therapeutic benefit experienced by a patient may be increased by administering one of the compounds described herein with another therapeutic agent (which also includes a therapeutic regimen). By way of example only, in a treatment for pain involving administration of one of the compounds described herein, increased therapeutic benefit may result by also providing the patient with another therapeutic agent for pain. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention together with inert or active compounds, or other drugs including wetting agents, flavor enhancers, preserving agents, stabilizers, humidity regulators, pH regulators, osmotic pressure modifiers, emulsifiers, UV-A and UV-B screening agents, antioxidants, depigmenting agents such as hydroquinone or kojic acid, emollients, moisturizers, for instance glycerol, PEG 400, or urea, antiseborrhoeic or antiacne agents, such as S-carboxymethylcysteine, S-benzylcysteamine, salts thereof or derivatives thereof, or benzoyl peroxide, antibiotics, for instance erythromycin and tetracyclines, chemotherapeutic agent, for example, paclitaxel, antifungal agents such as ketoconazole, agents for promoting regrowth of the hair, for example, minoxidil (2,4-diamino-6-piperidinopyrimidine 3-oxide), non-steroidal anti-inflammatory agents, carotenoids, and especially p-carotene, antipsoriatic agents such as anthralin and its derivatives, eicosa-5,8,11,14-tetraynoic acid and eicosa-5,8,11-triynoic acid, and esters and amides thereof, retinoids, e.g., RAR or RXR receptor ligands, which may be natural or synthetic, corticosteroids or oestrogens, alpha-hydroxy acids and a-keto acids or derivatives thereof, such as lactic acid, malic acid, citric acid, and also the salts, amides or esters thereof, or p-hydroxy acids or derivatives thereof, such as salicylic acid and the salts, amides or esters thereof, ion-channel blockers such as potassium-channel blockers, or alternatively, more particularly for the pharmaceutical compositions, in combination with medicaments known to interfere with the immune system, anticonvulsant agents include, and are not limited to, topiramate, analogs of topiramate, carbamazepine, valproic acid, lamotrigine, gabapentin, phenytoin and the like and mixtures or pharmaceutically acceptable salts thereof. A person skilled in the art will take care to select the other compound(s) to be added to these compositions such that the advantageous properties intrinsically associated with the compounds of the invention are not, or are not substantially, adversely affected by the envisaged addition.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, methods for treating diseases, disorders, conditions, or symptoms in a patient (e.g., a human or animal) in need of such treatment are presented herein, the methods comprising the step of administering to the patient an amount of a compound of the invention effective to reduce or prevent the disease, disorder, condition, or symptom, in combination with at least one additional agent for the treatment of said disorder that is known in the art.

### EXAMPLES

In a related aspect, therapeutic compositions having at least one novel compound of the invention described herein can be administered in combination with one or more additional agents for the treatment of any of the diseases, disorders, conditions, or symptoms described herein.

It is understood that the foregoing examples are merely illustrative of the present invention. Certain modifications of the articles and/or methods employed may be made and still achieve the objectives of the invention. Such modifications are contemplated as within the scope of the claimed invention.

### Example I

### Synthesis of Novel Organophosphonate Acetylcholinesterase Inhibitors

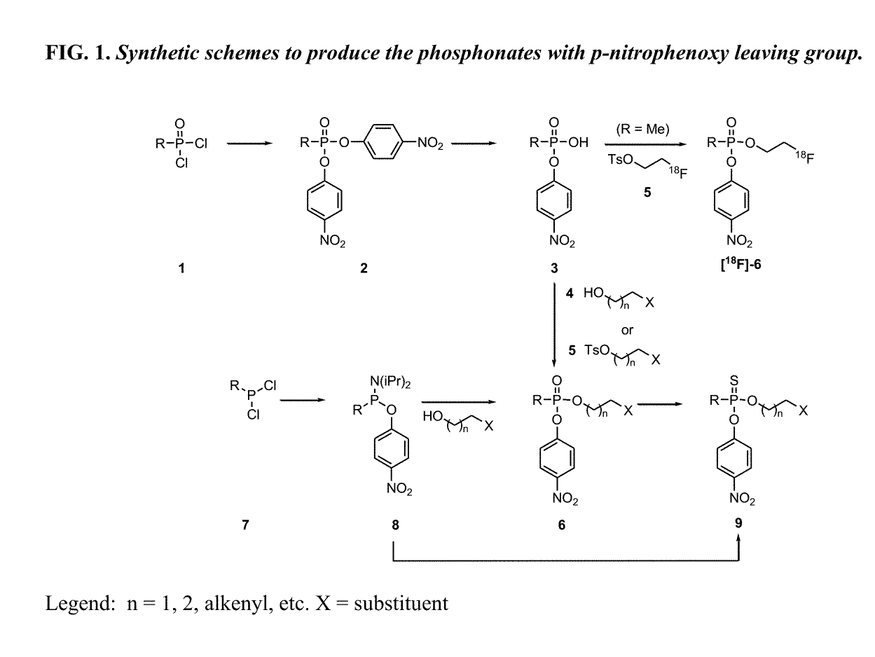

FIG. 1 depicts a synthetic scheme to prepare examples defined by Formula I of the invention in which the structures contain the leaving group Z=p-nitrophenoxy. An alkyl phosphonic acid bearing a p-nitrophenoxy ester 3 is prepared from an alkyl phosphonic dichloride, and used to prepare substituted alkoxy ester phosphonates 6 and substituted alkoxy ester phosphonothionates 9 (P═S). Structures found in the synthetic sequence in FIG. 1 utilize the p-nitrophenoxy ester (Z=p-NO₂—Ph—O) because this functional group is a leaving group in the reaction of organophosphorus compounds with certain biomolecules including cholinesterases [Fest, 1973; Eto 1974] while reducing volatility (as compared to Z═F) and increasing lipophilicity. The synthesis of analogs with Z=p-NO₂—Ph—O is described in Examples Analogs of 6 are also accessed via phosphonamidate (phosphorus-III) compounds such as 8 [Helinski,1991].

When the transformation of 3 to 6 (R═CH₃, n=1, X═F) is conducted with the fluorine-18 radioisotope of 2-fluoroethyl 4-methylbenzenesulfonate 5, the product formed is [¹⁸F]-6, the synthesis of which is described in Example VII and the utility described in Example VIII as a candidate biomarker tracer to detect AChE and profile AChE enzyme distributions in live and post mortem CNS and peripheral tissues.

### Example II

### In Vitro Acetylcholinesterase Inhibition

In this example, the in vitro acetylcholinesterase binding and inhibition of certain analogs were assessed. All solvents and reagents were reagent grade or better, used without any additional purification, and were purchased from Sigma-Aldrich (Milwaukee, Wis., USA). Electric eel acetylcholinesterase and recombinant human acetylcholinesterase were purchased from Sigma-Aldrich (Milwaukee, Wis., USA). Rat brain acetylcholinesterase was isolated and used in the assay as previously described [Thompson, 1989].

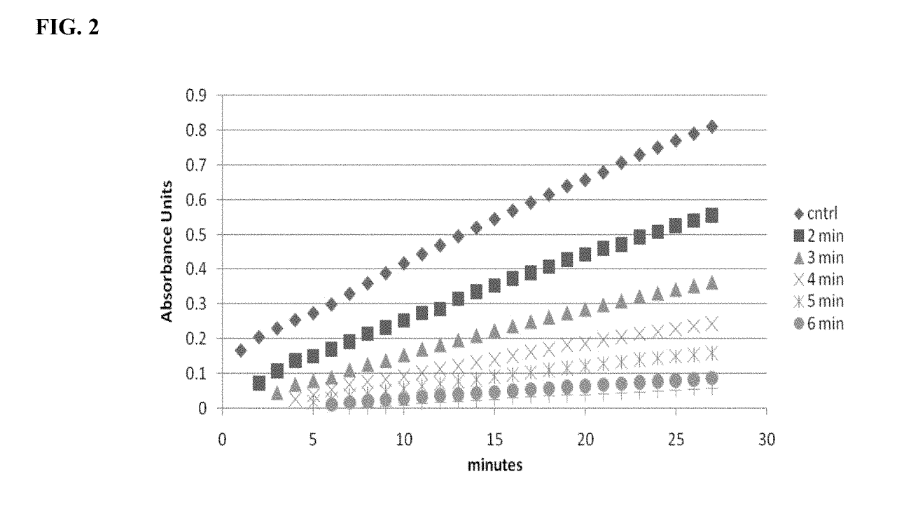

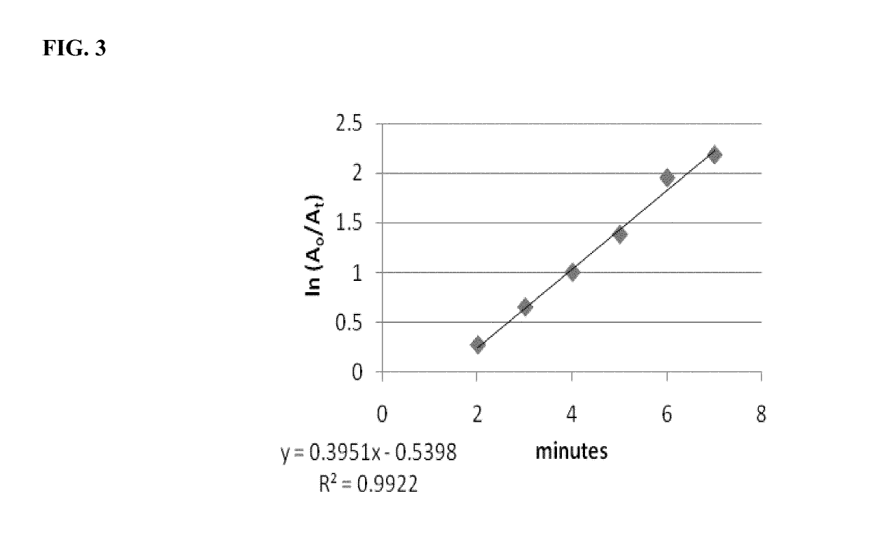

The results of these studies obtained by using established biochemical protocols within the art are shown in FIGS. 2 and 3, and demonstrate that compound 6 interacts with AChE in a specific manner through inactivation of acetylcholinesterase by covalent modification. Inactivation of electric eel acetylcholinesterase (EEAChE; an inexpensive cholinesterase commonly used for screening inhibitors) by compound 6 prevents the hydrolysis of the natural substrate acetylcholine with a bimolecular inhibition constant (kᵢ) of 5.90±0.15×10⁶ M⁻¹min⁻¹, which is comparable with paraoxon, a potent anti-AChE agent. The bromo (9: X═Br) and tosylate (9: X═OTs) P═O analogs also inhibited EEAChE with kᵢ values of 8.11±0.29×10⁴ M⁻¹min⁻¹ and 1.14±0.03×10⁵ M⁻¹min⁻¹, respectively, which are weaker inhibitors that fluoro analog 6. The phosphonothionate (P═S) analog 10 (X═Br) inhibited EEAChE with a kᵢ=1.14±0.03×10⁵ M⁻¹min⁻¹.

Compound 6 (R═Me, n=1, X═F) also inactivated recombinant human acetylcholinesterase (rHAChE) with kᵢ=7.51±0.21×10⁶ M⁻¹min⁻¹ and rat brain acetylcholinesterase (RBAChE) with kᵢ=6.11±0.25×10⁶ M⁻¹min⁻¹, which is similar to the inhibitory strength observed against EEAChE. Moreover, no reactivation of enzyme activity was observed after several hours following inhibition indicative of covalent modification of the active site serine as a likely mechanism.

An ethyl phosphonate analog (Formula I; Z═CH₃CH₂, R²═CH₂CH₂, X═Br, Y═S) inhibited EEAChE with a kᵢ=5.52±0.09×10⁴ M⁻¹min⁻¹ and RB AChE with a kᵢ=1.16±0.06×10⁶ M⁻¹min⁻¹.

### Example III

### Synthesis of Compounds 2 and 3

The synthesis of the precursor bis(p-nitrophenoxy) methyl phosphonate and key synthetic intermediate p-nitrophenoxy methyl phosphonic acid is illustrated below. The phosphonic acid 3 is a representative intermediate structure used to prepare analogs shown in Formula I.

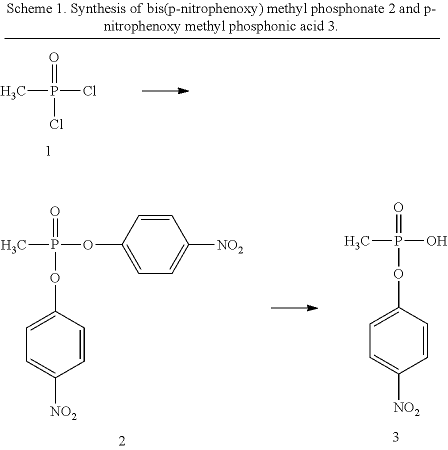

The Scheme 1 transformation serves as an example of the preparation of p-nitrophenoxy alkyl phosphonic acids leading to analogs of the invention. All solvents and reagents were reagent grade or better, used without any additional purification, and were purchased from Aldrich Chemical Company (Milwaukee, Wis., USA).

Synthesis of Compound 2. To methyl phosphonic dichloride (11.3 mmol) was added 4-nitrophenol (22.5 mmol) in CH₂Cl₂ (20 mL). The mixture was cooled in an ice-water bath and triethylamine (TEA; 45.1 mmol) in dry CH₂Cl₂ (5 mL) was added drop wise with stirring. TEA-HCl formed as the mixture stirred for 4 h at rt whereupon the reaction mixture was poured into 100 mL of ice cold water and extracted with CH₂Cl₂ (2×100 mL). The combined organic layers were dried over anhydrous Na₂SO₄, filtered and the solvent removed to yield crude bis(4-nitrophenyl) methylphosphonate 2 (Ghanem, 2007) that was purified on a short silica column using EtOAc:hexanes (3:7) and isolated as a pale yellow solid (3.49 g, 91%); ¹H NMR (500 MHz, CDCl₃) δ 8.22 (d, J=9.29 Hz, 4H), 7.37 (d, J=9.29 Hz, 4H), 1.95 (d, J=17.71 Hz, 3H); ¹³C NMR (500 MHz, CDCl₃) δ 154.53, 145.08, 125.88, 121.11, 12.62 (d, J_{CP}=144.25 Hz); ³¹P NMR (500 MHz, CDCl₃) δ 25.34; HRMS Calcd for C₁₃H₁₁N₂O₇P 338.0304; Found 339.0307 [(M+H)⁺].

Synthesis of Compound 3. To the bis(4-nitrophenyl) methylphosphonate (7.4 mmol) in CH₃CN (28.8 mL) was added 0.5 M LiOH (28.8 mL) drop wise using a pressure equalizing funnel over 20 min and stirred at rt for 1 h. The CH₃CN was removed under reduced pressure, and the aqueous solution extracted with CH₂Cl₂ (3×250 mL) to remove p-nitrophenol. The aqueous phase was then acidified with 3 N HCl to pH ˜0.5 and extracted with CH₂Cl₂ (3×250 mL). The organic phases were combined, and concentrated to give a crude semisolid that was re-crystallized using EtOAc:pentane (1:9). 4-Nitrophenoxy hydrogen methylphosphonate (3: R═Me) was isolated as a crystalline light brown solid (0.86 g, 54%); ¹H NMR (500 MHz, CDCl₃) δ 8.20 (d, J=9.13 Hz, 2H), 7.32 (d, J=9.16 Hz, 2H), 1.66 (d, J=16.69 Hz, 3H); ¹³C NMR (500 MHz, CDCl₃) δ 154.76, 144.81, 125.70, 121.26, 12.53 (d, J_{CP}=139.25 Hz); ³¹P NMR (500 MHz, CDCl₃) δ 31.14; HRMS Calcd for chemical formula C₇H₈NO₅P 217.0140; Found: 218.0188 [(M+H)⁺].

### Example IV

### Synthesis of Compound 6. (R═Me, n=1, X═F)

Scheme 2 illustrates an example of a synthesis of a Formula I compound as a p-nitrophenoxy, β-fluoroethoxy methylphosphonate (Formula I; where R═Me, n=1, X═F), which is prepared in this example by a method using carbodiimide coupling. In addition to serving as a Formula I structure, the product of this transformation, compound 6, is a non-radioactive standard of the radioactive agent [¹⁸F]-6 (Example VI, below) and an analog of the nerve agent, VX.

Solvents and reagents were reagent grade or better, used without any additional purification, and were purchased from Aldrich Chemical Company (Milwaukee, Wis., USA).

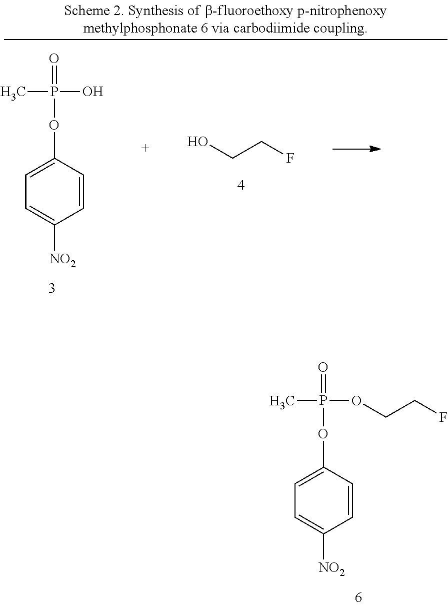

Compound 6. To 4-nitrophenyl hydrogen alkylphosphonate 3 (0.5 mmol) in dry CH₂Cl₂ (5 mL) was added 2-fluoroethanol 4 (0.5 mmol) and dicyclohexylcarbodiimide (0.9 mmol) at rt with stirring for 24 h. The reaction mixture was filtered through filter paper to remove N,N′-dicyclohexylurea, the filtrate diluted with CH₂Cl₂ (50 mL), washed with DI water (3×50 mL), and the CH₂Cl₂ layer dried (Na₂SO₄). Filtration of Na₂SO₄ and removal of the solvent yielded the crude product that was purified over silica using EtOAc:hexanes (6:4) to afford 2-fluoroethyl 4-nitrophenyl methylphosphonate 6 as a colorless sticky mass (76.3 mg; 58%): ¹H NMR (500 MHz, CDCl₃) δ 8.85 (d, J=9.27 Hz, 2H), 7.40 (d, J=9.27 Hz, 2H), 4.50-4.67 (m, 2H), 4.24-4.47 (m, 2H), 1.75 (d, J=17.85 Hz, 3H); ¹³C NMR (500 MHz, CDCl₃) δ 155.14, 144.75, 125.89, 121.11, 82.72, 81.35, 12.21 (d, J_{CP}=144.15 Hz); ³¹P NMR (500 MHz, CDCl₃) δ 29.33; ¹⁹F NMR (500 MHz, CDCl₃) δ −224.47; HRMS Calcd for chemical formula C₉H₁₁NFO₅P 263.0359; Found: 264.0434 [(M+H)⁺].

### Example V

### Microwave-Assisted Alkylation Synthesis of Compound 6

Scheme 3 illustrates an example of a synthesis of Formula I compounds in which p-nitrophenoxy, β-fluoroethoxy methylphosphonate (6; R═Me, n=1, X═F) is once again prepared but by a second method using microwave-assisted alkylation. The synthesis was executed in very high conversion and amenable to radiolabeling (see Example 6).

Solvents were reagent grade or better, used without any additional purification and were purchased from Aldrich Chemical Company (Milwaukee, Wis., USA). 2-Fluoroethyl 4-methylbenzenesulfonate 5 was purchased from Sinova Chemicals USA.

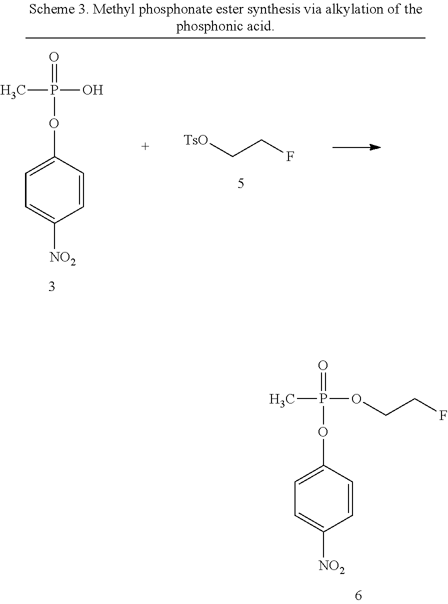 [SMILES: CP(=O)(O)Oc1ccc([N+](=O)[O-])cc1 ; Cc1ccc(S(=O)(=O)OCCF)cc1 ; CP(=O)(OCCF)Oc1ccc([N+](=O)[O-])cc1]

4-Nitrophenyl hydrogen methylphosphonate 3 (30 mg, 0.138 mmol) was taken up in 2 mL of dry CH₃CN in a 10 mL microwave vessel under an atmosphere of nitrogen gas. To this was added Cs₂CO₃ (22 mg, 0.069 mmol) and 2-fluoroethyl 4-methylbenzenesulfonate (0.026 mL, 0.151 mmol). The reaction mixture was stirred at 130° C. for 10 min at a power setting of 350-400 W in a microwave reactor after which the reaction was diluted with 5 mL of CHCl₃ to form a white precipitate. The precipitate was removed by gravity filtration and the filtrate concentrated to one-third of its original volume. The product was purified using preparative TLC (EtOAc:hexanes, 7:3) to afford 6 as an off-white solid (18.5 mg 51%). The spectral and physical data was identical to that reported in Example IV.

### Example VI

### Synthesis of Compound 10

Scheme 4 illustrates a synthesis of phosphonothionates 10, which are Formula I compounds in which the pi-bond is a thiophosphoryl, P═S group. In this example, methyl phosphonate 6 is converted to the corresponding thionate (P═S) 10 a P═O to P═S conversion using Lawesson's sulfurating reagent. Solvents and reagents were reagent grade or better, used without any additional purification, and were purchased from Aldrich Chemical Company (Milwaukee, Wis., USA).

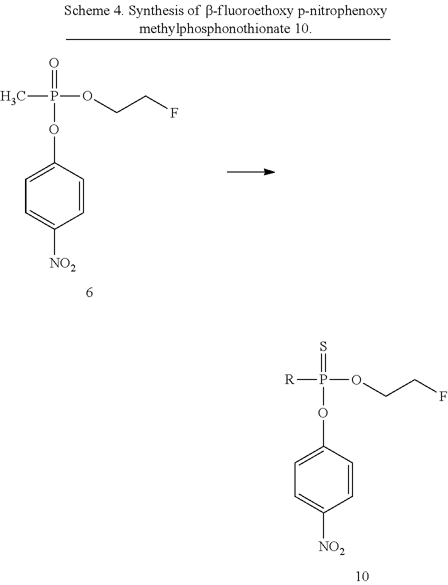

To compound 6 (0.2 mmol) in 3 mL of dry toluene was added 2,4-bis(4-methoxyphenyl)-1,3,2,4-dithiadiphosphetane-2,4-disulfide (Lawesson's Reagent; 0.1 mmol) and the reaction mixture brought to reflux for 3 h. The reaction was cooled to room temperature, filtered, triturated with 2 mL CHCl₃, and the filtrate concentrated and purified using preparative TLC (1:3, EtOAc:hexanes) to obtain O-2-fluoroethyl-O-p-nitrophenyl methyphosphonothionate 10 as a semisolid (35.7 mg; 64%): ¹H NMR (500 MHz, CDCl₃) δ 8.26 (d, J=9.27Hz, 2H), 7.34 (d, J=9.27 Hz, 2H), 4.49-4.66 (m, 2H), 4.24-4.48 (m, 2H), 2.11 (d, J=15 Hz, 3H); ¹³C NMR (500 MHz, CDCl₃) δ 154.99, 145.01, 125.43, 122.41, 82.70, 81.33, 66.35, 22.67 (d, J_{CP}=460 Hz); ³¹P NMR (500 MHz, CDCl₃) δ 96.15; ¹⁹F NMR 500 MHz, CDCl₃) δ −224.29; HRMS Cacld for C₉H₁₁FNO₄PS 279.0130; Found 280.0128 [(M+H)⁺].

### Example VII

### Radiochemical Synthesis of AChE Positron Emission Tomography (PET) Imaging Tracers

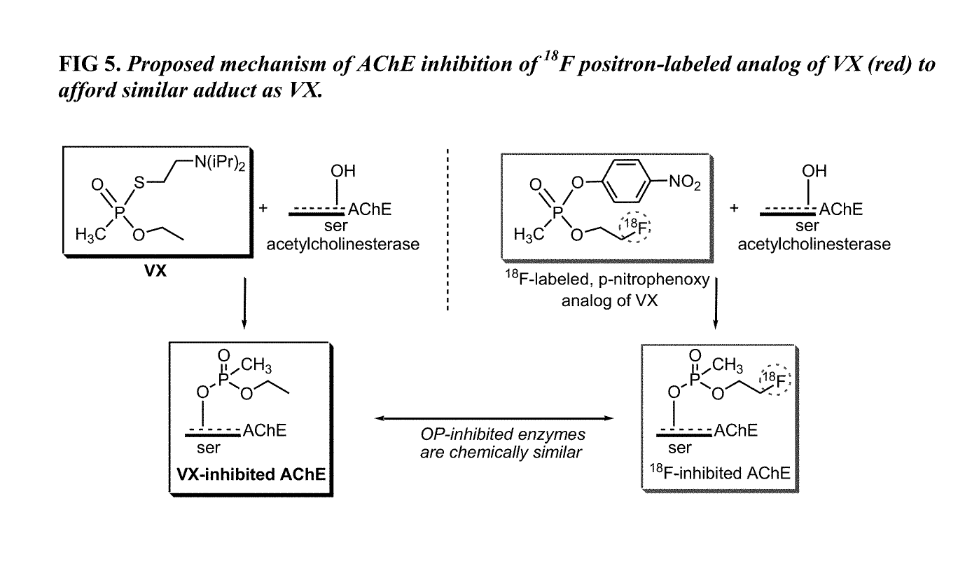

The synthesis (Scheme 5) and utility of a novel radiolabeled fluorine-18 ([¹⁸F]) variant [¹⁸F]-6 of the organophosphorus compound 6 is illustrated below. Radiolabeled analog [¹⁸F]-6 is a candidate biomarker tracer to detect AChE and profile AChE enzyme distributions in live and post mortem CNS and peripheral tissues. FIG. 5 illustrates the mechanisms of AChE inhibition by the nerve agent VX as well as compound [¹⁸F]-6.

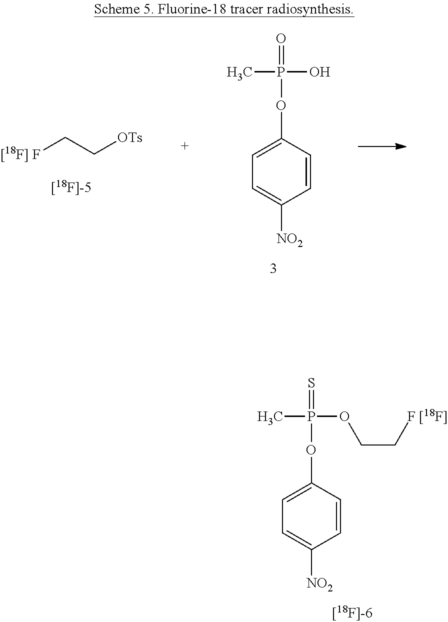

The Scheme 5 transformation serves as an example for fluorine-18 ([¹⁸F]) radiolabeling reactions to afford tracer [¹⁸F]-6 of the invention and is related to the chemical synthetic transformations described in Example V. Acetonitrile, ethyl acetate, hexanes and cesium carbonate (Cs₂CO₃) were reagent grade or better, used without any additional purification, and were purchased from Aldrich Chemical Company (Milwaukee, Wis., USA). USP grade phosphate buffered saline pH 7.4 was also purchased from Aldrich Chemical Company. High performance liquid chromatography (HPLC) was performed with a Waters (Milford, Mass.) 590 chromatograph.

The Scheme 5 [¹⁸F]-containing reagent, 2-[¹⁸F]fluoroethyl-1-(4-methyl)benezenesulfonate [¹⁸F]-5, was prepared according to literature methods [Herth 2009, Musachio 2005, Lu 2004], using a GE PETtrace medical cyclotron. Radioactive reagent [¹⁸F]-5 (0.05-0.25 mg) and acetonitrile (0.25 mL) were placed within a small Pyrex microwave reaction vessel that contained Cs₂CO₃ (3.3 mg), precursor 3 (6.2 mg), and a few molecular sieves. The vessel was sealed with a non-metal crimp top, and then placed in a Biotage Initiator 8 microwave reactor unit. The vessel was subjected to microwave radiation at 130° C., 400 W initial power, which then stabilized to 70 W, over the course of 10 min. Thereafter, the vessel and contents were allowed to cool to room temperature. The crude material within the vessel was purified by semi-preparative HPLC using radioactivity detection. The crude residue was diluted (2-5 mL) with a mixture of ethyl acetate:hexanes (3:1), chromatographed employing this same isocratic solvent system, using a Phenomenex column (250×10 mm, 100 Å silica phase) and a flow rate of 4 mL/min. The major radioactive peak containing [¹⁸F]-6 was collected beginning at ˜18.0 min elution time. Portions (2-4 μL) of this collected sample were evaluated by analytical HPLC for quality control (QC) assessments.

The QC analytical HPLC was performed with a Phenomenex column (250×4.60 mm, 100 Å silica phase), a solvent mixture of ethyl acetate:hexanes (3:2), flow rate of 1 mL/min and also UV (254 nm) and radioactivity detection. The QC HPLC radioactivity peak collected at ˜7.5 min possessed the same retention time as the non-radioactive (cold) ligand standard 6 (UV detection). The QC HPLC elution profile demonstrated that the tracer [¹⁸F]-6 material was greater than 98% pure.

The semi-preparative HPLC collected tracer fraction was processed by removal of solvent under a stream of nitrogen gas. The resultant residue was formulated as rodent (e.g., rat) doses by dissolving the residue in a mixture of 0.1 mL acetonitrile and 0.9 mL PBS pH 7.4. The average decay corrected radiochemical yield of [¹⁸F]-6 was ˜5% (n=17), based upon reagent [¹⁸F]-5.

To determine specific activity, a standard curve was constructed using the areas under the analytical HPLC 254 nm peak of compound 6 standard, in which known concentrations of 6 (for example, five or more concentrations at 1 mg/mL-0.01 mg/mL) were evaluated. Acetonitrile aliquots of either the non-radioactive standard or the tracer [¹⁸F]-6 were subjected to analytical HPLC (Phenomenex Luna column, 250×4.6 mm, 10 μm), eluted with 4:1 ethyl acetate:hexanes at 1.0 mL/min flow rate. The areas under the peak for tracer [¹⁸F]-6 were interpolated against the standard curve to determine the mass of [¹⁸F]-5 from which specific activity (radioactivity/mass; Ci/mmol) was calculated. The average specific activity of tracer [¹⁸F]-6 at the time of injection was calculated as ˜2,050 Ci/mmol (n=17).

### Example VIII

### Tracer Brain and Peripheral Imaging in Rodent Subjects: In Vivo Characteristics of Compound [¹⁸F]-6

As an example of the tracer of the invention, rodent (rat) quantitative in vivo PET imaging trials are presented employing the tracer compound [¹⁸F]-6 of Example VII. The in vivo PET imaging was performed in parallel with magnetic resonance (MR) and computed axial tomography (CAT, CT) imaging methods, in which the latter two imaging methods afforded anatomical tissue information for co-registration to the acquired tracer quantitative PET data. The co-registration of imaging data sets allows for the definitions of tissue regions of interest (ROIs) that possess various EAAT2 tissue densities (concentrations).

Four rodent imaging trials (A-D) were performed employing male Sprague-Dawley subjects. The rat subject ages were similar to those described in the most recent rat (Sprague-Dawley, amongst others) stereotaxic brain atlas [Paxinos 2007]. Thus, there were high confidence levels for the identification and definition of explicit brain cerebral fine tissue structures as regions of interest (ROIs) for quantitative analysis, and as a function of three-dimensional (3D) co-registration of cerebral soft tissue identification by MR scan analysis and landmark anatomical features from CAT data. Details of the four subject (A-D) imaging tracer trials are summarized in Table 1.

TABLE 1

| | | | Tracer | | Tracer | | |
|---|---|---|---|---|---|---|---|
| | Rat | Rat | [¹⁸F]-6 | Tracer | [¹⁸F]-6 | Compound | Challenge |
| | Subject | Subject | Specific | [¹⁸F]-6 | Dose | Pre-administered | dose |
| | Age | Weight | Activity | Dose | Volume | 10 min prior | volume |
| Trial | (days) | (g) | (Ci/mmol) | (mCi) | (mL) | (mg/Kg dose) | (mL) |
| A | 64 | 290 | 2,104 | 1.118 | 0.5 | None | None |
| B | 69 | 300 | 1,992 | 0.857 | 0.5 | Non-radioactive, | 0.5 |
| | | | | | | compound 6 (2.0) | |
| C | 102 | 370 | 2,098 | 1.208 | 0.6 | Paraoxon (0.030) | 0.5 |
| D | 90 | 470 | 1,997 | 0.876 | 0.6 | Echothiophate | 0.5 |
| | | | | | | (0.030) | |

The PET data were acquired with a Siemens Inveon microPET/CT scanner system (ca. 1.5 mm PET imaging spatial resolution). The rodent tail vein tracer [¹⁸F]-6 injection volumes (Table 1) used the tracer [¹⁸F]-6 dose formulation described in Example VII. Tracer [¹⁸F]-6 injections were followed by a 0.3 mL saline flush. The pre-administration (challenge, blocking) Trials B-D were accomplished by injection of the Table 1 pre-administered agents, given as tail vein injections 10 minutes prior to administration of the tracer and using dose formulations similar to that used for tracer [¹⁸F]-6. The pre-administered challenge agent evaluations included Trial B as the non-radioactive form of the tracer, e.g., compound 6 as described in Example V (2.0 mg/kg dose), Trial C as the organophosphorus agent paraoxon (0.030 mg/Kg dose), and Trial D as the organophosphorus agent echothiophate (0.030 mg/Kg dose).

PET imaging was performed northermic (37° C.) with rats under isoflurane anesthesia (1-1.5%). The dynamic PET data were acquired either for 120 or 180 min durations, beginning approximately 1 min after the time of injection of tracer [¹⁸F]-6. The PET data were reconstructed as 12 frames, 600 seconds per frame, for the 120 min scan times or as 18 frames, 600 seconds per frame, for 180 min scans. Magnetic resonance (MR) data were acquired with a Bruker Biospin 7-Tesla magnet multi-slice 2D FLASH (T2*-weighted gradient recall echo, TR=1528.3 msec, TE=7 msec, 256×256×50 voxels, 16 μm³ resolution). Computed axial tomography (CAT) data were acquired with a Siemens CT scanner in standard rat mode (80 kVp, 225 mA; 400 ms exposure, 194 steps×194 degrees, 97 micron isotropic resolution).

MR, CT and PET imaging data files were processed with AMIDE open source software [Loening 2003] (UCLA, Los Angeles, Calif.), version 0.9.0 (or later versions). MR and CT images were oriented as defined by Paxinos [2007]. Cranial landmarks of bregma and lambda were identified from the CT images. The X, Y, Z coordinates of imaging views were centered as bregma=origin of Trial A. Consistent landmark structures were iteratively co-registered and template fit against the cranial structures of the Trial A landmarks, and cross checked against cerebral soft tissues observed from the MR scan data. All PET scan data were decay time corrected and quantified with a phantom instrument calibration factor. Regional central nervous system tissue and peripheral tissue radioactivity is reported as Standardized Uptake Value (SUV) defined as: (MBq in the tissue region of interest/decay corrected injected dose at time=0)/body weight of the rat as Kilogram (Kg) [Innis 2007].

Each Trial A-D PET data sets were iteratively co-registered to respective CT skull data and fine adjustments were made using the Trial A PET data as a template. Central nervous system and peripheral organ tissue regions of interest (ROIs) were defined conservatively (well within the ROI volume size limits and locations) against their stereotaxic 3D locations [Paxinos 2007] and correlated with the MR tissue landmarks. The central nervous tissue ROIs are defined as follows: FrCtx as frontal cortex, MotCtx as motor cortex, CgCtx as cingulate cortex, CP as caudate-putamen, TH as thalamus, ME as mescencephalon referred to as the mid-brain, RE as the rhombencephalon otherwise referred to as the brain stem, and CE as cerebellum. ROI PET scan statistics were exported to Excel and graphs of SUV versus time were generated using GraphPad Prism software (La Jolla, Calif.).

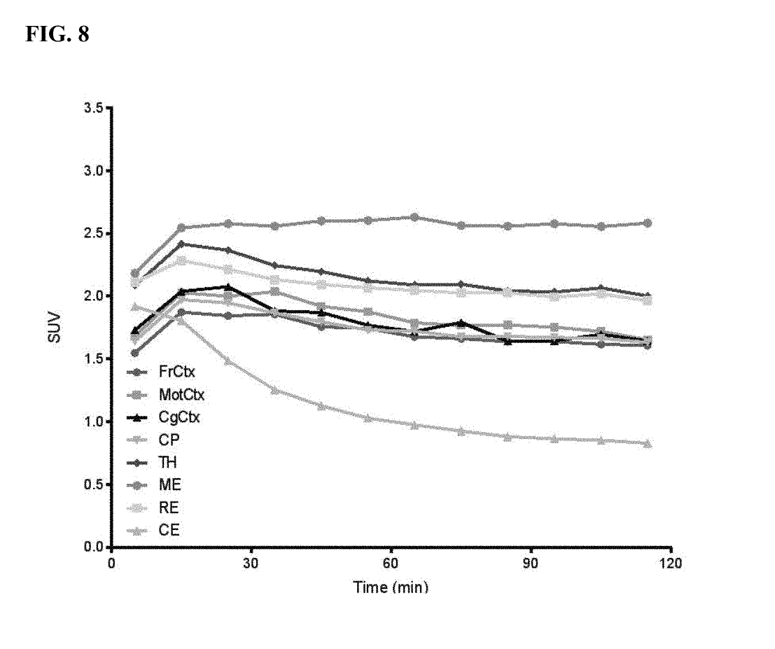

The tracer time-activity curves of FIG. 8, where only tracer [¹⁸F]-6 was administered (a baseline scan), show that the injected tracer [¹⁸F]-6 penetrates into brain, and at various times post injection, discrete radioactivity SUV signals per tissue ROIs are observed over the course of 0-120 minutes. The PET tracer [¹⁸F]-6 affords tissue radioactivity pharmacokinetic curves that are observed with uptake and a maximum. For the majority of the brain ROIs and with the exception of the cerebellum region, little, if any, tracer tissue washout occurs over the course of the 120 min evaluation period. Differential magnitudes of radioactive signals are observed as a function of brain tissue, especially when assessed at 120 min post tracer [¹⁸F]-6 injection. For example, high radioactive signals are found in the mesencephalon (ME), thalamus (TH), and rhombencephalon (RE). Somewhat lower radioactive SUV signals are observed in the frontal, motor and cingulate cortices (FrCtx, MotCtx and CgCtx; respectively), and also the caudate-putamen (CP). A low radioactive signal is observed in the cerebellum (CE) region.

All of these radioactivity SUV signals and their magnitudes are consistent with other findings that organophosphorus agents, such as tracer [¹⁸F]-6, have significant interactions with the enzyme acetylcholinesterase within these tissues [Jett 2007, Sidell 1992, Finkelstein 1988, Eto 1974, Fest 1973]. These enzyme-tracer [¹⁸F]-6 interactions include the formation of a covalent bond adduct between a serine residue within the enzyme acetylcholine binding site and the organophosphate tracer phosphorus atom [Jett 2007, Sidell 1992, Eto 1974, Fest 1973]. It is observed that tracer [¹⁸F]-6 possesses brain tissue distribution profiles that are similar to known rodent brain region distributions of acetylcholinesterase determined by other agents and alternative means of detection [Kickuchi 2007, Ryu 2005, Musachio 2002, Planas 1994, Tavitan 1993, Segal 1988, Biegon 1986]. Therefore, by the intravenous administration of tracer [¹⁸F]-6, acetylcholinesterase is detected in discrete central nervous system tissue ROIs as a function of known acetylcholinesterase distributions across live rat brain.

### Example IX

### In Vivo AChE Inhibition Studies

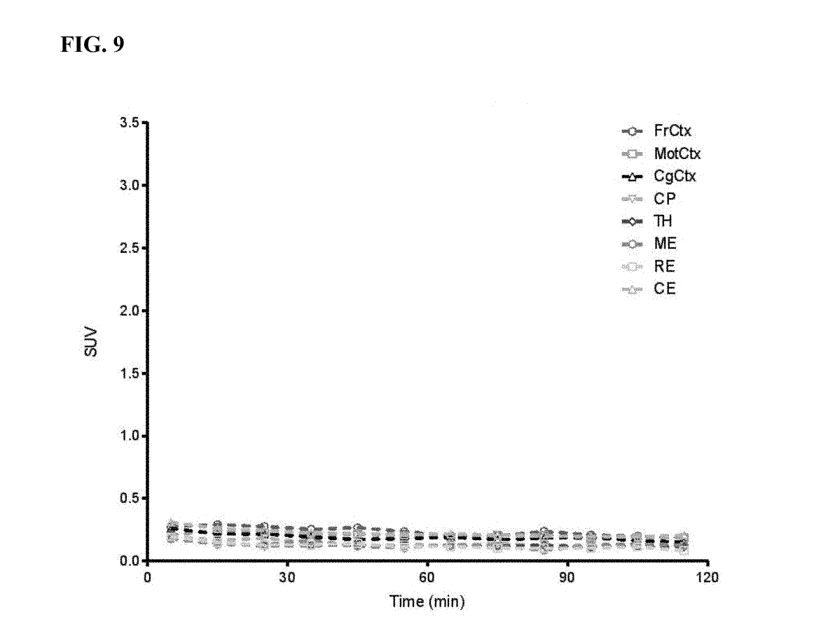

The outcome of the Trial B pre-administration challenge study, where compound 6 (2.0 mg/Kg dose) was administered 10 min prior to tracer [¹⁸F]-6 is shown FIG. 9. The data reveals a significant, greater than four-fold, reduction of SUV radioactivity signals across all brain tissue ROIs when compound 6 is administered in advance of tracer [¹⁸F]-6. Comparison of the FIG. 8 and FIG. 9 data provide evidence that tracer [¹⁸F]-6 is interacting with acetylcholinesterase, since it has been demonstrated in Example II that compound 6 is a potent in vitro, covalent inhibitor of acetylcholinesterase. Therefore, the pre-administration of compound 6 is thought to have blocked available acetylcholinesterase binding sites from the subsequently administered tracer [¹⁸F]-6. Since rat acetylcholinesterase brain distributions have been correlated to acetylcholinesterase distributions in primate brain [Hiraoka 2009, Kikuchi 2007, Shinotoh 2004, Blomqvist 2001, Volkow 2001, Koeppe 1999, Pappata 1996, Biegon 1986], and given that tracer [¹⁸F]-6 interacts with acetylcholinesterase in rat brain, then tracer [¹⁸F]-6 is thought capable of detecting acetylcholinesterase in primate brain.

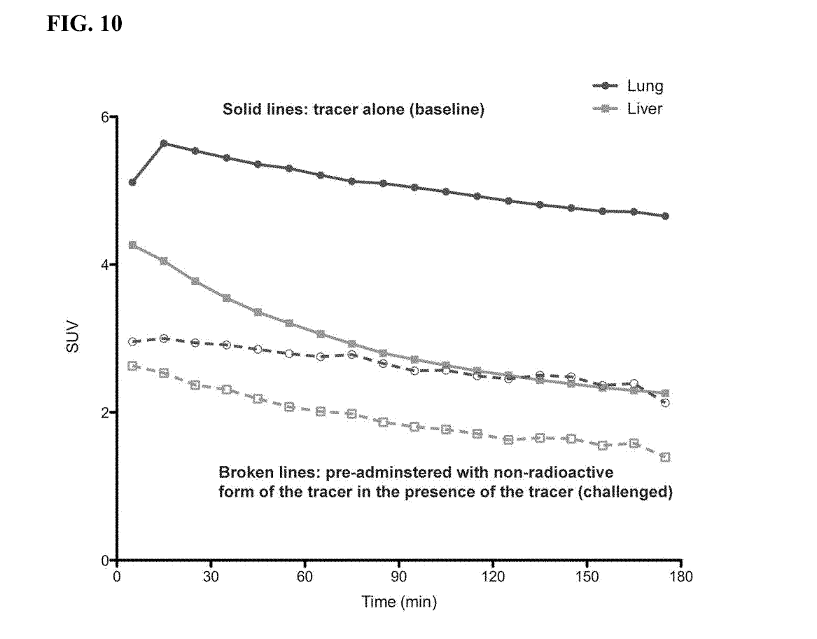

A similar comparison of Trial A versus Trial B tracer [¹⁸F]-6 has been made in select peripheral tissues, including the lung and liver regions, as shown in FIG. 10. In this study, the lung and liver pharmacokinetic radioactivity signals are shown for Trial A as solid lines, lung as solid circles and liver as solid squares; and also for Trial B as broken lines with open circles for lung and open squares for liver. For the Trial A solid line curves, it is observed that tracer [¹⁸F]-6 has tissue radioactivity signal maxima and then a slow radioactivity tissue wash out. The Trial B broken line curves also possesses radioactivity signal maxima and slow tissue radioactivity wash out.

A comparison of the Trial A and Trial B curves of FIG. 10 reveal that during all times of the Trial B, there are significantly reduced radioactivity signals in lung and liver tissues when tracer [¹⁸F]-6 has been administered after the challenge agent compound 6; and in relative comparison to the tracer alone (baseline) pharmacokinetic radioactivity signal curves of Trial A. From the FIG. 10 data, it is thought that since acetylcholinesterase is found in lung and liver [Sidell 1992, Eto 1974, Fest 1973], then tracer [¹⁸F]-6 is interacting with this enzyme in these tissues. Since rat acetylcholinesterase peripheral tissue distributions have been correlated to acetylcholinesterase distributions in primate brain [Hiraoka 2009, Kikuchi 2007, Shinotoh 2004, Blomqvist 2001, Volkow 2001, Koeppe 1999, Pappata 1996, Biegon 1986], and given that tracer [¹⁸F]-6 detects acetylcholinesterase in rat peripheral tissues, then tracer [¹⁸F]-6 is thought capable of detecting acetylcholinesterase in primate peripheral tissues. Additionally, since carboxylesterase enzymes of the A-esterase variety are also found in these same tissues [Jakanovic 2001, Maxwell 2001, Satoh 1998, Maxwell 1992], it is thought that tracer [¹⁸F]-6 is also interacting with carboxylesterase enzymes in these peripheral tissues.

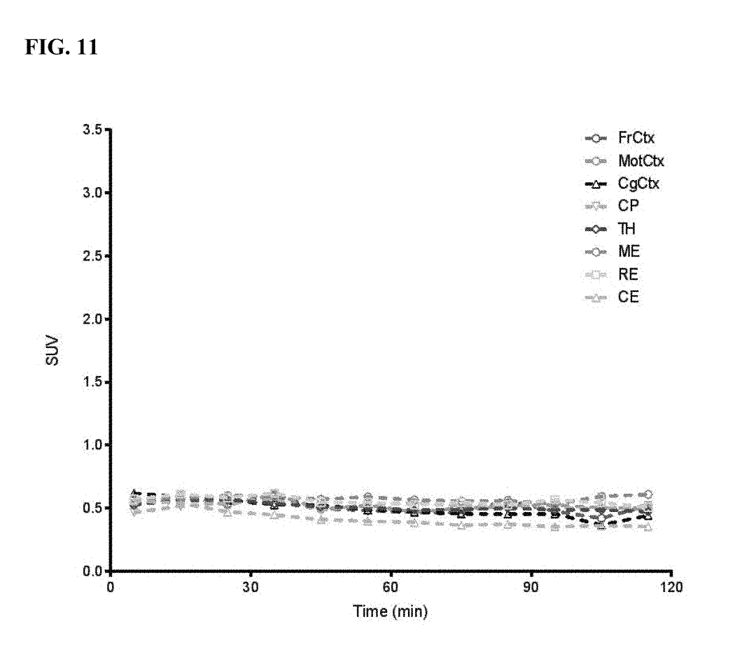

The results of the Trial C pre-administration challenge study, where the known organophosphorus compound paraoxon (0.030 mg/Kg dose) was administered 10 min prior to tracer [¹⁸F]-6, is shown FIG. 11. The data reveals a significant reduction of SUV radioactivity signals across all brain ROIs when paraoxon is administered in advance of tracer [¹⁸F]-6. The comparison of the data of FIG. 8 versus FIG. 11 provides evidence that tracer [¹⁸F]-6 is interacting with acetylcholinesterase in these tissues, since it has been demonstrated earlier that paraoxon possesses potent inhibitory binding profiles of acetylcholinesterase [Houze 2010, Kardos 2000]. Therefore, the pre-administration of paraoxon is thought to have blocked available acetylcholinesterase binding sites from the subsequently administered tracer [¹⁸F]-6. Additionally, the comparison of the pharmacokinetic curves of FIG. 9 versus FIG. 11 reveal that the outcomes from the Trial D pre-administration with paraoxon result in reduced radioactivity signals in all brain regions that are similar in magnitude to the Trial B radioactivity signal curves of FIG. 9. These observations support the conclusion that tracer [¹⁸F]-6 has the property of detecting brain tissue that has been prior exposed to an organophosphorus agent; for example, the acetylcholinesterase inhibitor agent paraoxon.

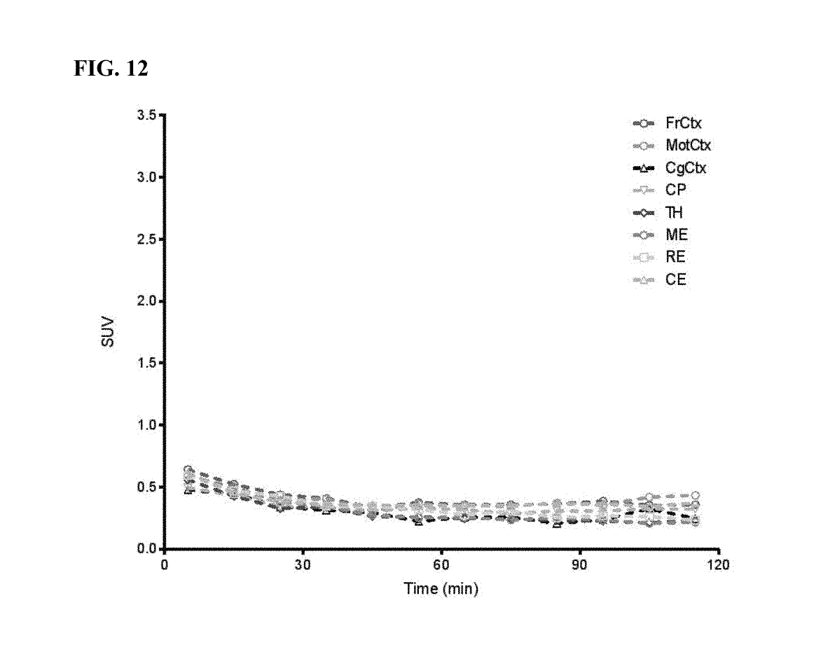

The outcome of the Trial D pre-administration challenge study, where the known organophosphorus compound echothiophate (0.030 mg/Kg dose) was administered 10 min prior to tracer [¹⁸F]-6, is shown FIG. 12. The data reveals a significant reduction of SUV radioactivity signals across all brain ROIs when echothiophate is administered in advance of tracer [¹⁸F]-6. The comparison of the data of FIG. 8 versus FIG. 12 provides evidence that the ability of tracer [¹⁸F]-6 to penetrate into brain is significantly reduced when echothiophate is administered in advance of tracer [¹⁸F]-6. These observations support the conclusion that tracer [¹⁸F]-6 has the property of detecting tissues that have been prior exposed to an organophosphorus agent; for example, the peripheral acetylcholinesterase inhibitor agent echothiophate [Mutch 1995].

### REFERENCES

Biegon A, Wolff M (1986) Quantitative histochemistry of acetylcholinesterase in rat and human brain postmortem. Journal of Neuroscience Methods, 16:39-45.
Blomqvist G, Tavitan B, Pappata S, Crouzel C, Jobert A, Doignon I, DiGiamberardino L (2001) Quantitative measurement of cerebral acetylcholinesterase using [¹¹C]physostigmine and positron emission tomography. Journal of Cerebral Blood Flow and Metabolism, 21:114-131.
Eto M (1974) Organophosphorus pesticides; organic and biological chemistry. CRC Press; Cleveland, Ohio.
Fest C, Schmidt K J (1973) The chemistry of organophosphorus pesticides; reactivity, synthesis, mode of action, toxicology. Springer Verlag; New York, N.Y.
Finkelstein Y, Wolff M, Biegon A (1988) Brain acetylcholinesterase after acute parathion poisoning: a comparative quantitative histochemical analysis postmortem. Annals of Neurology, 24:252-257.
Funakai Y, Kato M, Iwata R, Sakurai E, Sakurai E, Tashiro M, Ido T, Yanai K (2003) Evaluation of the binding characteristics of [5-¹¹C-methoxy]donepezil in the rat brain for the in vivo visualization of acetylcholinesterase. Journal of Pharmacological Sciences, 91:105-112.
Ghanem E, Li Y, Xu C, Raushel F M (2007) Characterization of a phosphodiesterase capable of hydrolyzing EA 2192, the most toxic degradation product of the nerve agent VX. Biochemistry, 46: 9032.
Helinski J, Dabkowski W, Michalski J (1991) O-p-Nitrophenyl N,N-diisopropyl-P-methylphosphonoamidite: novel difunctional P(III) reagent in oligonucleoside methylphosphonate synthesis containing 4-nitrophenoxy group. Tetrahedron Letters 32: 4981-4.
Herth M M, Kramer V, Rosch F (2009) Synthesis of novel WAY 100635 derivatives containing a norbornene group and radiofluorination of [¹⁸F]AH1.MZ as a serotonin 5-HT_{1A} receptor antagonist for molecular imaging. Journal of Labelled Compounds and Radiopharmaceuticals, 52:210-207.
Hiraoka K, Okamura N, Funaki Y, Watanuki A, Tashiro M, Kato M, Hayashi A, Hosokai Y, Yamasaki H, Fujii T, Mori W, Yanai K, Watabe H (2009) Qunatitative analysis of donepezil binding to acetylcholinesterase using positron emission tomography and [5-¹¹C-methoxy]donepezil. Neurolmage, 46:616-623.
Houze P, Mager, D E, Risede P, Baud F J (2010) Pharmacokinetics and toxicodynamics of pralidoxime effects on paraoxon-induced respiratory toxicity. Toxicological Sciences, 116:660-672.
Innis R B, Cunningham V J, Delforge J, Fujita M, Gjedde A, Gunn R N, Holden J, Houle S, Huang S-C, Ichise M, Iida H, Kimura Y, Koeppe R A, Knudsen G M, Knuuti J, Lammertsma A A, Laruelle M, Logan J, Maguire R P, Mintun M A, Morris E D, Parsey R, Price J C, Slifstein M, Sossi V, Suhara T, Votaw J R, Wong D F, Carson R E (2007) Consensus nomenclature for in vivo imaging of reversibly binding radioligands. Journal of Cerebral Blood Flow and Metabolism, 27:1533-1539.
Jakanovic M (2001) Biotransformation of organophosphorus compounds. Toxicology, 166:139-160.
Jett D A (2007) Neurological aspects of chemical terrorism. Annals of Neurology, 61:9-13.
Kardos S A, Sultatos L G (2000) Interactions of the organophosphates paraoxon and methyl paraoxon with mouse brain acetylcholinesterase, Toxicological Sciences, 58:118-126.
Kikuchi T, Okamura T, Fukushi K, Takahashi K, Toyohara J, Okada M, Zhang M-R, Irie T (2007) Cerebral acetylcholinesterase imaging: development of the radioprobes. Current Topics in Medicinal Chemistry, 7:1790-1799.
Koeppe R A, Frey K A, Snyder S E, Meyer P, Kilbourn M R, Kuhl D E (1999) Kinetic modeling of N-[¹¹C]methylpiperidin-4yl-proprionate: alternatives for analysis of an irreversible positron emission tomography tracer for measurement of acetylcholinesterase activity in human brain. Journal of Cerebral Blood Flow and Metabolism, 19:1150-1163.
Loening A M, Gambhir S S (2003) AMIDE: a free software tool for multimodality medical image analysis. Molecular Imaging, 2:131-137.
Lu S-Y, Chin F t, McCarron J A, Pike V W (2004) Efficient O- and N-(β-fluoroethylation)s with NCA [18F]β-fluoroethyl tosylate under microwave-enhanced conditions. Journal of Labelled Compounds and Radiopharmaceuticals, 47:289-297.
Maxwell D M, Brecht K M (2001) Carboxylesterase: specificity and spontaneous reactivation of an endogenous scavenger for organophosphorus compounds. Journal of Applied Toxicology, 21:S103-S107.
Maxwell D M (1992) The specificity of carboxylesterase protection against the toxicity of organophosphorus compounds. Toxicology and Applied Pharmacology, 114:306-312.
Musachio J L, Flesher J E, Scheffel U A, Rauseo P, Hilton J, Mathews W B, Ravert H T, Dannals R F, Frost J J (2002) Radiosynthesis and mouse brain distribution studies of [¹¹C]CP-126,998: a PET ligand for in vivo study of acetylcholinesterase. Nuclear Medicine and Biology, 29:547-552.
Mutch E, Kelly S S, Blain P G, Williams F M (1995) Comparative studies of two organophosphorus compounds in the mouse. Toxicology Letters, 81:45-53 1995.
Pappata S, Tavitan B, Traykov L, Jobert A, Dalger A, Mangin J F, Crouzel C, DiGiamberardino L (1996) In vivo imaging of human cerebral acetylcholinesterase. Journal of Neurochemistry, 67:876-879.
Paxinos G, Watson C (2007) The Rat Brain in Stereotaxic Coordinates, 6^{th} edition. Elsevier; Burlington, Mass.
Planas (1994) Rat brain acetylcholinesterase visualized with [¹¹C]physostigmine. NeuroImage, 1:173-180.
Ryu E K, Choe Y S, Park E Y, Paik J-Y, Kim Y R, Lee K-H, Choi Y, Kim S E, Kim B-T (2005) Synthesis and evaluation of 2-[¹⁸F]fluoro-CP-118,954 for the in vivo mapping of acetylcholinesterase. Nuclear Medicine and Biology, 32:185-191.
Satoh T, Hosokawa H (1998) The mammalian carboxylesterases: from molecules to functions. Annual Reviews of Pharmacology and Toxicology, 38:257-288.
Segal M, Greenberger V, Israeli M, Biegon A (1988) A correlation between regional acetylcholinesterase activity in rat brain and performance in spatial task. Behavioural Brain Research, 30:215-219.
Shinatoh H, Fukushi K, Nagatsuka S-I, Irie T (2004) Acetylcholinesterase imaging: its use in therapy evaluation and drug design. Current Pharmaceutical Design, 10: 1505-1517.
Sidell F R, Borak J (1992) Chemical warfare agents I. Nerve agents. Annals of Emergency Medicine, 21:865-871.
Tavitan B, Pappata S, Planas A M, Jobert A, Bonnot-Lours A, Crouzel C, DiGiamberardino L (1993) In vivo visualization of acetylcholinesterase with positron emission tomography. NeuroReport, 4:535-538.
Thompson C M, Frick J A, Natke B C, Hansen L K (1989) Preparation, analysis, and anticholinesterase properties of O,O-dimethyl phosphorothioate isomerides. Chemical Research in Toxicology, 2: 386-91.
Volkow N D, Ding Y-S, Fowler J S, Gatley S J (2001) Imaging brain cholinergic activity with positron emission tomography: its role in the evaluation of cholinergic treatments in Alzheimer's dementia. Biological Psychiatry, 49:211-220.

### Example X

### Exemplary Compounds of the Invention

Exemplary compounds of the invention include, without limitation, the following species derived from Formula I, wherein substituent Z is p-nitrophenol, substituent R¹ is methyl or ethyl, substituent Y is oxygen or sulfur, substituent R² group is a substituted ethyl, isopropyl or cyclohexyl group, and substituent X is defined below:

A. Methyl ethoxy phosphonate Series:

X═H, ethyl 4-nitrophenyl methylphosphonate; X═F, 2-fluoroethyl 4-nitrophenyl methylphosphonate; X═Cl, 2-chloroethyl 4-nitrophenyl methylphosphonate; X═Br, 2-bromoethyl 4-nitrophenyl methylphosphonate; X═I, 2-iodoethyl 4-nitrophenyl methylphosphonate; X═tosylate, 2-[(4-methylbenzenesulfonyl)oxy]ethyl 4-nitrophenyl methanephosphonate.

B. Ethyl ethoxy phosphonate Series:

X═H, ethyl 4-nitrophenyl ethylphosphonate; X═F, 2-fluoroethyl 4-nitrophenyl ethylphosphonate; X═Cl, 2-chloroethyl 4-nitrophenyl ethylphosphonate; X═Br, 2-bromoethyl 4-nitrophenyl ethylphosphonate; X═I, 2-iodoethyl 4-nitrophenyl ethylphosphonate; X=tosylate, 2-[(4-methylbenzenesulfonyl)oxy]ethyl 4-nitrophenyl ethane-1-phosphonate.

C. Methyl isopropoxy phosphonate Series:

X═H, 4-nitrophenyl propan-2-yl methylphosphonate; X═F, 1-fluoropropan-2-yl 4-nitrophenyl methylphosphonate; X═Cl, 1-chloropropan-2-yl 4-nitrophenyl methylphosphonate; X═Br, 1-bromopropan-2-yl 4-nitrophenyl methylphosphonate; X═I, 1-iodopropan-2-yl 4-nitrophenyl methylphosphonate; X=tosyl, 1-[(4-methylbenzenesulfonyl)oxy]propan-2-yl 4-nitrophenyl methanephosphonate.

D. Ethyl isopropoxy phosphonate Series:

X═H, 4-nitrophenyl propan-2-yl ethylphosphonate; X═F, 1-fluoropropan-2-yl 4-nitrophenyl ethylphosphonate; X═Cl, 1-chloropropan-2-yl 4-nitrophenyl ethylphosphonate; X═Br, 1-bromopropan-2-yl 4-nitrophenyl ethylphosphonate; X═I, 1-iodopropan-2-yl 4-nitrophenyl ethylphosphonate; X=tosyl, 1-[(4-methylbenzenesulfonyl)oxy]propan-2-yl 4-nitrophenyl ethanephosphonate.

E. Methyl 4-cyclohexymethyl phosphonate Series:

X═H, 4-methylcyclohexyl 4-nitrophenyl methylphosphonate; X═F, 4-(fluoromethyl)cyclohexyl 4-nitrophenyl methylphosphonate; X═Cl, 4-(chloromethyl)cyclohexyl 4-nitrophenyl methylphosphonate; X═Br, 4-(bromomethyl)cyclohexyl 4-nitrophenyl methylphosphonate; X═I, 4-(iodomethyl)cyclohexyl 4-nitrophenyl methylphosphonate; X=tosyl, (4-{[methyl(4-nitrophenoxy)phosphoryl]oxy}cyclohexyl)methyl 4-methylbenzene-1-sulfonate.

F. Ethyl 4-cyclohexymethyl phosphonate Series:

X═H, 4-methylcyclohexyl 4-nitrophenyl ethylphosphonate; X═F, 4-(fluoromethyl)cyclohexyl 4-nitrophenyl ethylphosphonate; X═Cl, 4-(chloromethyl)cyclohexyl 4-nitrophenyl ethylphosphonate; X═Br, 4-(bromomethyl)cyclohexyl 4-nitrophenyl ethylphosphonate; X═I, 4-(iodomethyl)cyclohexyl 4-nitrophenyl ethylphosphonate; X=tosyl, (4-{[ethyl(4-nitrophenoxy)phosphoryl]oxy}cyclohexyl)methyl 4-methylbenzene-1-sulfonate.

G. Methyl ethoxy thionophosphonate (P═S) Series:

X═H, ethyl 4-nitrophenyl methyl(sulfanylidene)phosphonite; X═F, 2-fluoroethyl 4-nitrophenyl methyl(sulfanylidene)phosphonite; X═Cl, 2-chloroethyl 4-nitrophenyl methyl(sulfanylidene)phosphonite; X═Br, 2-bromoethyl 4-nitrophenyl methyl(sulfanylidene)phosphonite; X═I, 2-iodoethyl 4-nitrophenyl methyl(sulfanylidene)phosphonite; X=tosyl, 2-[(4-methylbenzenesulfonyl)oxy]ethyl 4-nitrophenyl P-methyl sulfanephosphonite

### All Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Other embodiments are within the claims.

What is claimed is:

1. A compound represented by the following formula 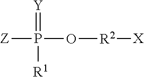
or a salt or ester thereof, wherein
R¹ is methyl;
R² is a branched, straight chain or cyclic alkyl;
X is selected from fluorine and alkoxy;
Y is selected from oxygen or sulfur; and
Z is a leaving group selected from the group consisting of fluorine, nitrile, thiol, thiocholine, substituted phenols and heterols, alcohols, cholines, and substituted amines and amides.

2. The compound of claim 1, wherein said Z is p-nitrophenoxy and X is fluorine.

3. The compound of claim 1, wherein said compound is selected from the group consisting of 2-fluoroethyl 4-nitrophenyl methylphosphonate 1-fluoropropan-2-yl 4-nitrophenyl methylphosphonate; 4-(fluoromethyl)cyclohexyl 4-nitrophenyl methylphosphonate; and 2-fluoroethyl 4-nitrophenyl methyl(sulfanylidene)phosphonate.

4. The compound of claim 1, wherein said fluorine is fluorine-18.

5. The compound of claim 1, wherein said alkoxy comprises carbon-11.

6. The compound of claim 1 in combination with a pharmaceutically acceptable excipient.

7. A method of synthesizing a tracer compound represented by the following formula 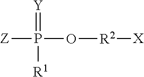
or a salt or ester thereof, wherein
R¹ is methyl;
R² is a branched, straight chain or cyclic alkyl;
X is fluorine-18;
Y is selected from oxygen or sulfur; and
Z is a leaving group selected from the group consisting of fluorine, nitrile, thiol, thiocholine, substituted phenols and heterols, alcohols, cholines, and substituted amines and amides;
comprising the steps of
reacting an alkylphosphonic dichloride with two equivalents of p-nitrophenol in the presence of a base to form a bis[p-nitrophenoxy] alkylphosphonate;
performing monohydrolysis to yield a p-nitrophenoxy alkylphosphonic acid; and
reacting said p-nitrophenoxy alkylphosphonic acid with cesium carbonate and radiolabeled [¹⁸F] beta-fluoroethyltosylate to yield said tracer compound.

8. A method of detecting a cholinesterase comprising the steps of
contacting a cholinesterase with a tracer compound represented by the following formula 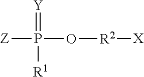
or a salt or ester thereof, wherein
R¹ is methyl;
R² is a branched, straight chain or cyclic alkyl;
X is fluorine-18 or carbon-11;
Y is selected from oxygen or sulfur; and
Z is a leaving group selected from the group consisting of fluorine, nitrile, thiol, thiocholine, substituted phenols and heterols, alcohols, cholines, and substituted amines and amides; and
detecting said tracer compound with a radiographic scanner.

9. The method of claim 8, wherein said radiographic scanner is a positron emission tomography scanner.
